(19) United States
(12) Patent Application Publication
(10) Pub. No.: US 2023/0245728 A1
(43) Pub. Date: Aug. 3, 2023

(54) Process for Dimensional Manipulation using Key Fractal Elements

(71) Applicant: Gregory M. Friedlander, Mobile, AL (US)

(72) Inventor: Gregory M. Friedlander, Mobile, AL (US)

(21) Appl. No.: 17/591,087

(22) Filed: Feb. 2, 2022

(51) Int. Cl. G16C 20/50; G06F 30/10

(52) U.S. Cl. CPC G16C 20/50; G06F 30/10

(57) ABSTRACT

This patent describes application of key fractal elements (KFE) of AuT (the science) inherent in the fractal design of dimension for dimensional manipulation based on fractal underpinnings of the universe. The process is applicable at all dimensional levels for defining and using fractal transitions. AuT defines the bit required for this logic to work (positive and negative result) and its operation in the universe. The iterated equations primarily discussed are fpix=−1\hat{ }x+2x(—1)\hat{ }(x−1), an equation generating the denominator of pi and made up and derived from underlying iterated equations, n+1 which drives changes in fpix and 2f(n)\hat{ }(2\hat{ }n) the observed compression function the results of which are called ct states. There are multiple potential models of lower ct states, dimensional states, but at the ct3-ct4 interface, the model is relatively accurate and involves f-series exponential compression about a central axis: 2:1 F-series overlap of two spiral arms which fold and unfold to compresses and decompress ct states organized in exponential quantities based on interactional exchanges of ct states toward or away from compression, these exchanges being referred to generally as absorption where they are added to a matrix, and spew where they exit a matrix. The applications cover all of practical science, but can be summarized by the resulting conclusion that all controlled actions are dimensional manipulation and AuT delivers the most efficient path.

### PRIORITY STATEMENT

This application claims priority based on the following USA filings:

Feb. 2, 2022: EFS ID 44890696 Application No. 63/305,415

Feb. 3, 2021 EFS 41840137 U.S. Pat. No. 3,145,752

Mar. 27, 2021 EFS 42205201 Application No. 63/162,147

May 20, 2021 EFS 42780031 Application No. 63/191,149

This is a CIP/Bypass application of the following applications:

Oct. 30,2021 Publication Number: 2020211087342

Int'l Application Number: PCT US2020/058333

May 6, 2021 U.S. Pat. No: 17,291,632 USA EFS ID 42650366

May 21, 2020 U.S. Pat. No: 16,880,255 5/21/20 EFS: 39507295

Aug. 2, 2019 U.S. Pat. No: 16,530,339 efs id: 36769989

Aug. 20, 2019 U.S. Pat. No: 16,552,481 Efs: 36993126

### TECHNICAL FIELD

This Patent applies to applications of a new model of quantum mechanics.

### BACKGROUND ART

This patent involves the application of a new model which replaces fundamental understandings of dimension and force.

### SUMMARY OF THE INVENTION

Fractal equivalence exists at every level which allows for modeling any process or structure based on higher and lower order structures. This model needed to be developed and applications needed to be developed. The result is a better understanding and categorizing and use of the features of the universe.

### BRIEF DESCRIPTION OF THE DRAWINGS

FIG. 1 shows compression

FIG. 2 Poynting interpretations of AuT

FIG. 3 AuT Plasma Fulcrums at the electron-proton level

FIG. 4 AuT plasma fulcrum alignment at the atomic level

FIG. 5 Complex Atoms

FIG. 5a A complex plasma core

FIG. 6 Carbon through a fractal lens

FIG. 7 Carbon Rings and electron pairing

FIG. 8A carbon helium core as Silicon

FIG. 8a A neon atom

FIG. 8b Argon collapsed

FIG. 8c Argon expanded

FIG. 8d Helium

FIG. 8e Lithium ion

FIG. 9 Plasma Canon

FIG. 10 Magnetic Repulsion

FIG. 11 Li-ion battery modeling top view

FIG. 12 Li-ion battery side view of FIG. 11

FIG. 13 Composite Shear dimensional modification

FIG. 14 composite shear with sheets

FIG. 15 composite shear with plugs

FIG. 16 composite shear screws

FIG. 17 represents relative size

FIG. 18 shows the effect of post time change compared to size.

FIG. 19 shows an octane molecule.

FIG. 20 shows a chlorophyl molecule.

### DESCRIPTION OF THE EMBODIMENTS

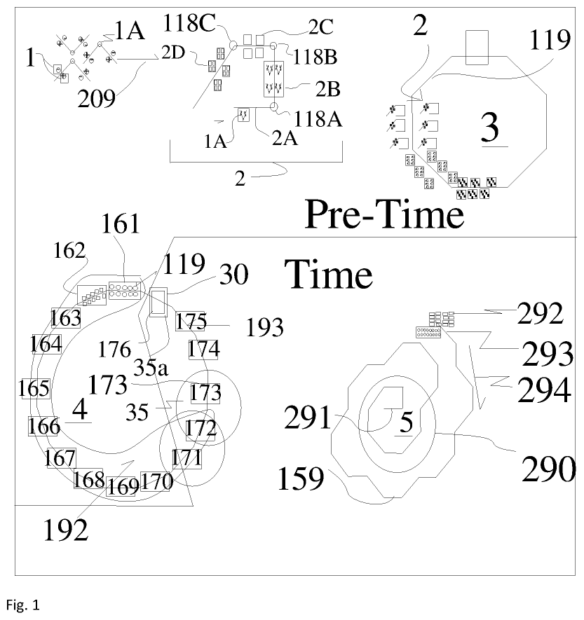

FIG. 1 shows staged compression between fpix and the minimum size black hole based on the equation 2f(n)\hat{ }2\hat{ }n.

Photon elements are believed to be made up of ct4t11, just as electron elements are believed to be 5.4 ct4t12 states (at the ct3-ct4 transitional level, these are also shortened to “t12 states”). Rather than using photon elements and electron elements, the more accurate fractal states designations are used understanding the exact ct states might change as reasonable experimentation improves the understanding of pretime states.

Fractal equivalence is differentiated from actual size. If an atom was collapsed, a zero-energy state in terms of thermodynamics or a perfect fractal in the language of AuT it might look like spiral drawings.

T12 states as used herein can be thought of as a more specific definition of Bloch electrons. While fulcrums are identified as separate features, the existence of a central ct state group, even if part of the core ct states would satisfy the conceptual framework of balance even if the separation turns out to be less specific than indicated.

11) designing at least one of: chemistry (reaction; fuel; carbon capture; rare earth combinations) based on fractal chemical and molecular structure of the at least one matrix; biology (DNA, ATP, biological design, and function); physics including titanic event prediction and control; quantum gravity and time, black holes, dark energy, wave particle duality, etc.); Energy capture, generation, and storage; fission; Quantum Computing using the non-pretime change and pretime changing with non-pretime changing or amount of pretime change to deliver or withhold information; Concentrating pre-time elements; qubit function and design utilizing KFE; Using ct states with different amounts of pre time change for relative work; Fusion by removing non-compressive information to get fusion.

Time is a stop frame effect of invisible pre-ct4t11 dimensional changes, energy is pretime change, the concept of a particle being in multiple places at once (pretime) giving it the Ct4t11 particles are in multiple places at once (pretime) giving them Archimedes wheel (AW) effect of waves.

Quantum change in dimension scales an absolute or best usable value of “Planck or Electromagnetic” time (PT or EMT). Quantum measurements include replacing Planck length with quantum ct state changes at at least one ct state compression level. The method includes tracking 1) absolute time and 2) utilizing the rate of absolute change for time keeping, reconciling electromagnetic time (EMT) with absolute time or quantum change includes using existing clock models to incorporate the mathematics of pre-time change to improve the results.

Pretime changes can be used as data for processing, refining the precise parameters of the dimensional features giving rise to EMT time mathematically and tracking these changes, recognizing that while an absolute definition of change exists; time is a function of multiple dimensional parameters that change incrementally with quantum change.

AuT includes utilizing, to the extent possible, the rate of absolute change for time keeping with physical structures; and determining rates of change at different locations in the universe which involves averaging the movement of large structures in the solar system or galaxy; and reconciling atomic clock frequencies with underlying dimensional version of time, the dimensional features of time themselves.

AuT includes treating AuT fpix changes as plus and minus. There are multiple ways in the electronic spectrum where the plus or minus result can be sampled or applied as with fuse length, compression vs decompression; abs vs spew; pretime and post time change. The absolute definition of change at ct1 is a quantum count common to all points in the universe, “absolute time” or more accurately “absolute change” or “AuT dimensional change.”

The relationship of absolute time to traditional time involves determining rates of change at different locations in the universe which involves averaging the movement of large structures in the solar system or galaxy.

AuT involves using the better understanding of frequency and pre-time change to design and use existing frequency-based systems to improve the results.

Measurement: Using dimensional change in place of time to measure what would otherwise be time-based measurements increases the accuracy.)

Communication and Time

AuT involves using the relationships of ct state exchange along KFE features and information capture and release to generate more efficiently, use, store and transport ct matrix results, not just energy, but all dimensional manipulation.

Manipulation includes using changes in pretime (pre ct4t11) ct states for determining pretime change for at least one ct state and basing communication or computing results on those pretime changes. By knowing the amount of pretime change on both ends in the information provided, communications can be made in a time free environment and the smaller the informational ct state accessed, the less time dependent it is although the overall amount of information decreases. Using smaller pretime states means that more information can be sent, for example, if information is sent in ct4t11 states within a single ct4t12 states there are 10 bits of ct4t11 for each one of the ct4t12. If electrons are used, there are 5 times 10 ct4t11 states for each electron (approximately) in a pair so that there are 50 states doing the same computing and/or communications as the single electron.

Time is the result of stop frame animation effects, changes are in fractal building blocks of electrons, ct412 states which, in turn, are made up of 10 ct4t11 states, typically viewed as photons. Ct4t11 states are made up of ct4t10 states which are made up of ct4t9 states and so on all the way back to ct1 states which are mathematical bits. The electron ct4t12 states and lower components (ct4t11 states) are in multiple locations and states for any measurable time and appear relativistic but only from a perspective of time. By dimensional analysis they remain Newtonian. Focusing on ct state pretime locations, quantum computing is done without or with at least less “probability” which suggests a “false randomness” which is replaced with specific “pretime locational or net AuT state change where AuT state changes are the positive or negative states in which ct1 states find themselves which are reflected in fractally significant positive or negative states in higher compression states. Pretime locations are locations, where a particle exists from a pre-time environment as seen from the perspective of time. Since these can be very significant for any period of time (practical measurements of 10\hat{ }44 of these changes per second is modeled based on the partial analysis of the speed of computing using this method is exponentially higher than with other computers.

QC-Pretime Transistors

A (pretime transistor) PTT is, essentially, a triode; substituting pretime change features, ct4t12 and lower for electromagnetic output. Like a traditional “vacuum triode,” the control of information into the grid and the resulting readings from drops between the cathode and anode (plate) and control of the heater are all critical to its operation. Pretime locations are used for computing in a post-time environment.

Multiple PTT(s) can be reconciled to determine pretime changes of qubits, and to take these pretime changes and arrange them in such a way as to get interpretive results.

In a matrix, you can get comparison of wavelengths, whether the same or by comparing different length wavelength at the same time. Individual ct states within what post time would be considered wave structures can be analyzed for changes to be used for computing, multiple ct states can be used to compare pretime changes so that the changes pretime can be organized to interpret in a post-time environment.

Molecular Pumps

This positron coming off an otherwise collapsed neutron is the reason that the absorption of the proton (the source of positive charge) is the same as the spew (unfolding) of the electron which is otherwise 1/1800th the size of the entire proton.

This positron provides a unique qubit because it is positively charged and locked in place on the proton. Focus on securing the place of the electron in qubit applications is not as significant where focus is on the positron which is necessarily fixed to a much more easily managed proton and which moves already has secured ionic forms of Li in Li-ion batteries. This relationship is easy to manipulate, relatively speaking, as electromagnetic:

1) To utilize the positrons as qubits it is important to develop holding procedures to maintain individual atoms in place. Semi-conductor structures are considered an excellent target because they are already used to control ion movement.

2) Building the structures and populating them with atoms to study and control reactions for different positive ions or finding commercially available semi-conductors which incorporate usable atoms already-Li-ion batteries and solar panels are good targets as they are already doped with usable atoms.

3) Develop the best methods for pushing the pump arms. The positron and electron arms are targets as natural handles, but the science will also focus on changes in the proton (reservoir) and on the fulcrum where the positron and electron come together, possibly using the positron itself as a fulcrum. This arrangement of features has never been identified before and therefore could not have been targeted.

4) Determine best energy wavelengths to interact with the elements, especially the positron (with or without changing the electron potentially). Any element can be a qubit, the smaller the element the more “pre-time” change is incorporated, the larger the easier to control. This model allows for a small positron, held in place by a large proton (relatively/1800 times larger).

5) Getting quantum readings using the position as the qubit and testing simple reactions where manipulation of the different elements is included in controlling and initiating the reactions.

6) The use of lasers to lower atom temperatures might be used to target this interface (positron to electron) to tap it in the same way as super-cooled atoms would otherwise are cooled.

7) The charge interface is a source of multiple transitions between photon level features and one target of the inquiry would be to test this interface where charge is established as a source of single photons on demand.

8) Target moving pretime change states outward in matrix and post pretime change inward.

By targeting both the interaction of the electron and positron and the positron itself, quantum features may be addressed without expensive and complex electron cubit cooling which is required in other applications.

The ability to trigger and control any type of chemical or atomic (nuclear) reaction using a newly identified atomic feature is significant since it has never been targeted before.

Since you eliminate the holding features required in other quantum computers by using the already secured proton positron significant savings in quantum computer qubit design are possible.

Time is stop frame animation arising from pre-time dimensional changes, those being below the level of electromagnetic spectrum (largely ct4t12 and lower) where our perception of change begins.

The positron and electron arms are targets as natural handles, but the science will also focus on changes in the proton (reservoir) and on the fulcrum where the positron and electron come together, possibly using the positron itself as a fulcrum. This arrangement of features has never been identified before and therefore could not have been targeted. Determine best energy wavelengths to interact with the elements, especially the positron (with or without changing the electron potentially). Any element can be a qubit, the smaller the element the more “pre-time” change is incorporated, the larger the easier to control. This model allows for a small positron, held in place by a large proton (relatively/1800 times larger). The charge interface is a source of multiple transitions between photon level features and one target of the inquiry would be to test this interface where charge is established as a source of single photons on demand targeting both the interaction of the electron and positron and the positron itself, quantum features may be addressed without expensive and complex electron cubit cooling which is required in other applications.

The arrangement of t12 states changes in a pretime environment these changes can be targeted as bits using either observations or reactions to “radiations” to bring the pretime changes into a post time interpretation environment. Using words like radiation or wave-particle duality confuses the discussion, but these are used to ease the reviewer into the new science.

The “wave” portion of wave-particle duality is the pretime change viewed from a post time perspective. These changes are not restricted by light speed because they occur below the compression state of light.

Stop frame animation is the right analogy. The smaller the increments of change between opening the aperture of the camera, the less jerky the movement. If one were to take a slow change stop frame animation and film it against the background of a fast frame animation, there would be a shadow, a wave, in the background.

1. Primary Particles

The electron count need not be the same as the proton count under the AuT atomic model, indeed such a result above the level of Neon is contraindicated. Just as the neutron count become based on what is needed for fractal balance of the Neutron backbone, so too is the electron cloud about the proton core one of balance and not identity.

Why Neutrons, Protons, Electrons and Photons as the primary atomic states directly above the level of time? Photons appear to be ct4t11 states, open by exponential math, but made up of 10ct4t10 states. Electrons are ct4t13 states made of 10 ct4t12 states. Electrons in the sense that we use the word are ½ of an electron pair, 5ct4t12 states. Note that the ct4t11 to ct412 transition gets folded into the operational movement of the electron. 10 aligned ct4t11 states exists as a transition, it isn't seen as a separate particle, presumably because it is, by exponential math (even exponent) closed. Ct4t13 to ct4t14 is lost observationally. The proton and the neutron are both made up of 10 ct5t15 states, the only difference being the imperfectly closed proton has a positron which can complete an electron pair. So out of ct 10, 11, 12, 13, 14, 15 and 16, in the “post time” observational frame you have 3 closed states (12, 14, 16) which are built from 3 open states (11, 13 and 15). CT4t10 is pretime and seen only as a part of a net effect at the ct4t11 photon scale.

One suggestion is that observed particles up to the Neutron level are composed of the paired odd and even exponent. In this case the photon is a ct4t11 composite of ct4t10 states, the electron a ct4t13 composite of ct4t12 states, and the proton a ct4t15 composite of ct4t14 states. The neutron can be seen as different because it is not a composite because the states have folded together to create the next unit of compression for ct5 transition states.

2. Semi-Conductors (SC)

SC are improved with better chemistry and electromagnetic function and structural placement of components. At pre-atomic scales neutron pumps, targeting different AuT plasma centers, balanced core structures; lines of absorption and spew; alignment of spirals; controlled absorption and spew of lower information states along fractal lines; and using AuT insulator conductor focusing at different ct states (scales of compression) allow for better traditional energy generation and more direct absorption of radiation under AuT principles of defining energy in terms of changing pretime states.

AuT is targeting KFE changes like Atomic doping to match spiral transition forms to increase transitions or limit dendrite formation; introduce additional pretime change at spiral defined locations to dissolve electron shells as a specific target as opposed to an unintended effect, utilizing the back and forth action of shared information between ct states and shifting expansion of plasma centers and ct composite states as information pumps, particularly for quantum computing.

AuT insulators are basically ct state matrices which allow the passage of a lower ct state which balances a higher ct state which is at least mostly constrained from passing through the AuT insulator. AuT antennae are matched states based on common KFE features, particularly pretime features scaled at posttime levels.

All Capture at whatever level of compression and in whatever AuT medium (metal electron clouds, ct4t11 or 12 clouds, etc) likely occur with unpaired similar positron style ct states exchanging information with electron style ct states, that is an exchange of absorption and spew, possibly in series to slow down higher energy particles, as it is likely to happen in the cloudiness of outer electrons in metal antennae and as such this capture can be targeted with great specificity in reactions, solar, radiation protection, absorption and dispersal and the like by controlling the absorption and spew characteristic relative to one another of the ct states to be affected, whether pretime, atomic or molecular.

3. Chemistry

AuT redraws atomic structure. Examples appear in the priority document, but some relevant changes are referred to here as exemplary.

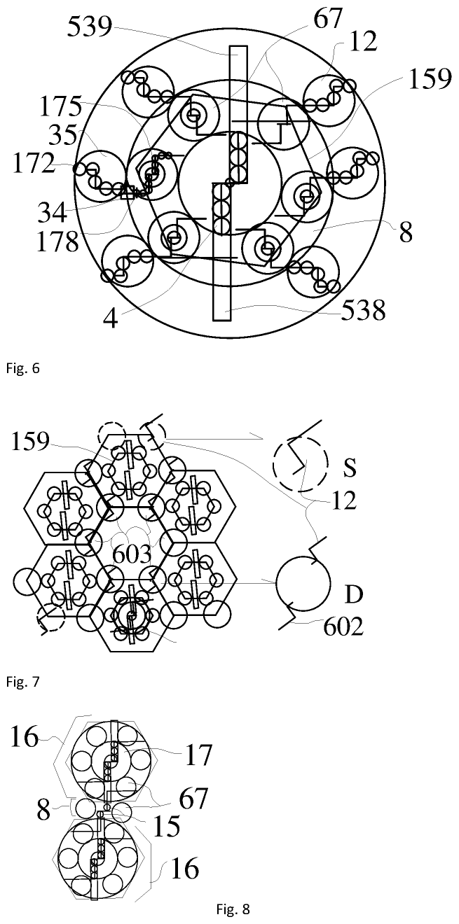

FIG. 6 shows a nanoscale view (ct4t12 level) of a Carbon ring compound. Showing the neutrons 4 as fractally aligned in the center to form a backbone represented by engines 538 and 539, the protons 8 around them made up of 10 ct4t15 states 175 electrons (only shown for an exemplary proton) which shares a positron 34 extending from broken ct4t15 state 178 with electron 35 which is like electrons 12 only designated separately so that the details can be observed, the f-series lines within the electrons 12 and 35 represent the two ct4t11 arms making up the two halves of a complete ct4t13 electron; the fractal area for the protons reflected by the fractally equivalent sizes to get the proper ratios in the periodic table and consistent with f-series (golden ratio) changes means that compared to a neon atom with the same area, there is room for six sided spacing as shown by the carbon hexagon 159 which spacing is responsible for the six sided structures of carbon. Carbon has a balanced proton core with a fractal hexagon 159, which may be repeated at different levels as shown in FIG. 6 and duplicated in ring compounds; varied with different ring cores as shown by the dna bases which have larger and smaller central features around differing fractal atom combinations, but fractally scaled as shown to hold a 5 or 6 unit ring structure, a form of centralized fulcrum for balancing multiple matrices comprising atoms.

The arrangement of protons within the hexagon 159 or other fractal structures for other atoms may be varied to allow for different bonding structures (double, triple, etc electron pairings with varying distances between proton openings to the neutron backbones. All atoms and molecules derive from and show the fractal underpinnings although the amount of exposed core and the fractal structure of the core will vary. Dimensional variations are represented in two dimensions; at lower and higher dimensions these fractal qualities are maintained according to the equations set out that give rise to them, primarily starting with fpix and determinations of prediction and manipulation using KFE; falling under the term dimensional manipulation in the claims, can be done following this simple method at more complex levels.

KFE includes control of where absorption and spew is generated within atomic structures and subatomic structures and where it is discharged into matrices which includes the concept of engines.

KFE “engines” focus absorption or spew of ct state exchanges which otherwise likely burst from the surface of atoms like volcanic eruptions from volcanos and solar flares from the sun. Neutron engines 538 and 539 are balanced.

Lithium engines might be unbalanced, the absorption and spew would be different exhibiting higher charge and reaction features. Engines appear likely to focus absorption and spew but are not the only places where it would occur in an atom.

4.a Reactions:

All reactions can be modeled and carried out using KFE features of at least one first AuT matrix (AM) and at least one second AuT matrix to maximize transitions through the exchange of ct states to get specific end results when the first AM and second AM exchange information, the AuT substitute for interactions, especially collisions, of particles.

4.b Fuel:

Reacting fuels using KFE to maximize the release of pretime informational change. spiral/exponential/centrally destabilized pathways; directly mining ct4t11 trapped in a fuel AuT matrix and stabilizing the resulting as by flushing the fuel AM with low pretime change ct4t11 to balance the release of the high pretime change ct4t11.

The case of boron fuels vs. octane fuels is instructive. If at least one compound or molecule generating water while oxidizing in boron distributed around boron atoms or BOH atoms. The enhanced pressure effect is seen in water developed in the octane-oxygen reaction. Control of proximity around KFE would enhance greatly the pressure created. Changes can be made around the octanes to enhance the water atom pressurization using KFE as fractal modeling and this enhanced reaction technique can be applied to any chemistry or biology question.

FIG. 7 shows fractal transition from the carbon to the six sided carbon ring showing the interaction of items 159 to form the ring structure shown. Bonds can occur with electrons 12. Hydrogen can bond to unpaired electrons which are otherwise bound through positrons as shown in other Figures. Here electrons paired are shown as an electron pair D made of electron 12 and a bonding electron 602. Absorption between the proton positron and the paired or unpaired electrons hold the atoms together. This S-D (single or double) bonding in Carbon reflects the release of positron bonds in favor of spew from electron pairs (pairs are ct4t13 states). Internal electron pairs 603 act as the AuT plasma for the ring compound. The unique hexagonal carbon ring structures can be seen serving the purpose of encouraging sharing of ct4t11 states within structures leading to magnetism in iron.

Structural relationships within comparable structures showing pretime locations of any electromagnetic signal can be captured and dispersed using chemicals arranged in appropriate structural fractal combinations and with dispersing or absorbing fractal structures. Absorption and spew of t11 states have similar features under this modeling targeting radar different wavelengths (tracking, targeting, etc) can be addressed with same chemical and fractal alignment.

FIGS. 5 and 5a show views of the entire atom in terms of FIGS. 5 and 5a showing the center with a matrix of expanded plasma. Just as the line between Hex1 and Hex2 is different and offset from the line between C1 and C2 so too the ct states to alter these may well need to be different and offset and this variation may have to change over the life of the time adjusting these in order to change the central or outer AuT Plasma Centers for reaction, catalyst action or otherwise.

Two different complex atoms are shown here, Xenon (N=77) and Radon (N=136). Isotopes are ignored, although in AuT, isotopes are poorly designated since the proton count is secondary. The combined carbon care is considered superior to the Argon net core despite the equivalence because the combined core allows for the 5.5 Carbon fractal size to end at a net of 11 Neutrons in the center allowing for balance, although the exact nature of the “shared” neutron (instead of 6 times 2 it would be 5×2 with a shared neutron, perhaps suspended and balanced within the plasma CP1. The general layouts are given without the specific numbers of neutrons in each Carbon backbone, the key being that each arm be balanced with a corresponding arm.

The similarities to political signs are coincidental and irrelevant.

In FIGS. 5 and 5a Neutron backbones C1 and C2 with two He Neutron Cores Hex2, although as shown these may be on either end. For continuity reasons having these compressed in the center is logical and balanced. The remaining neutrons are connected along the top (odd numbered from C3-C11 (not all are numbered to prevent the drawing from being unclear) and those at the bottom are even numbered C2-C12 for Xenon. CP1 is the plasma in this case between both HEX1 and 2 and C1 and C2. The top plasma centers can be numbered CP2t through CP6t and the bottom plasma centers CP2b-CP6b. For the more complex atoms with a left and right side, CP21-CP61 and CP2r-CP6r for the plasma components and C13-17 and C14-18 for the left and right-side carbon neutron backbones. FIG. 5a shows a complex plasma core of the type believed to be present in rare earth elements giving all larger atoms (those above Xenon) their unique catalytic qualities. There are 2 Carbon positrons 34 on either side where electrons 12 are found, there is a top Helium Neutron backbone Hex1 and bottom Neutron backbone Hex2 between the carbon backbones, and neutron to proton plasma 617 between neutrons throughout, but there is an expanded neutron backbone plasma 623 which is comparable to item 603 in FIG. 7. These cores along with the extended backbones allow for the total absorption and spew for the extended neutron backbones and associated proton cores to more easily shift position and this can be targeted in designing alloys and catalysts at this scale and at fractally equivalent scales in molecules.

AuT Plasma centers have a resting phase and an observational phase, the observation phase for Proton-Electron plasma centers is created by expanding the distance between protons and electrons, typically with microwave type radiation which is to say ct4t11-12 states with pretime change features which match those of plasma to allow an exchange of high pretime change ct4t11 states with those which have less pretime change and/or an increase in the amount of ct4t11-12 within the plasma center and/or higher compression with ct4t11-12 of the area of lower compression (lower than the plasma) around the plasma center.

Under AuT bonding occurs at the proton level for molecular interactions in the sense that opening within the proton matrix allow for information from openings in other atoms to be shared. It is also observed that a part of the process is the positrons of the protons release the electrons so they may be paired as free ct4t13 states with the necessary absorption and spew coming through the proton openings. The overall performance is equally controlled by the neutron backbone.

While the protons and electrons are responsible for bonding, the neutron backbone of the atom defines the movement of information, particularly with rare earth elements because of the expanding Neutron Backbone engines.

Xenon and Radon are shown. The Milky Way is a fractal equivalent of Radon. Expanded Neutron cores 15 are responsible for the changing operational features of RE atoms, in this case extended from the core sharing within all those neutrons between c1 and c2 for the close up, c1, c2, c3 and c4 for Xenon to those between c1, c2, c13 and c14, c3 and c4 at the core of radon.

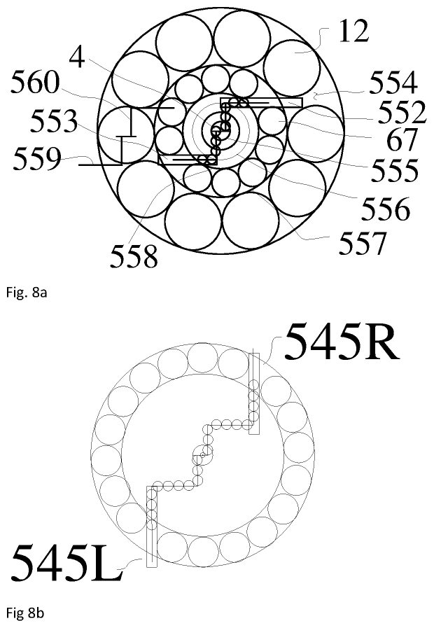

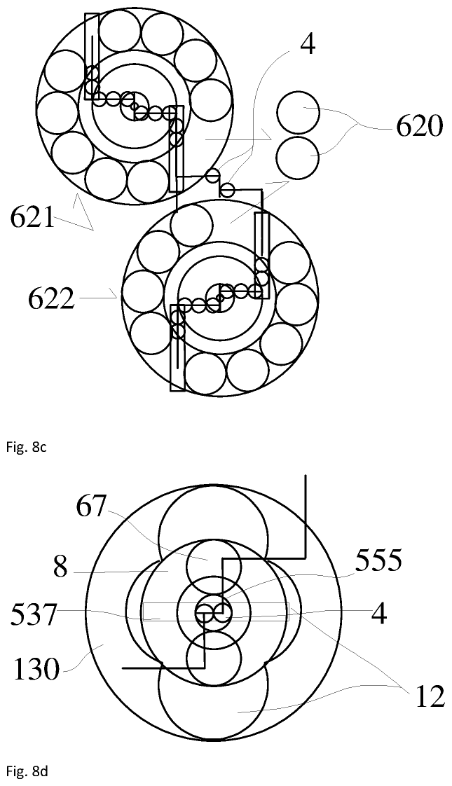

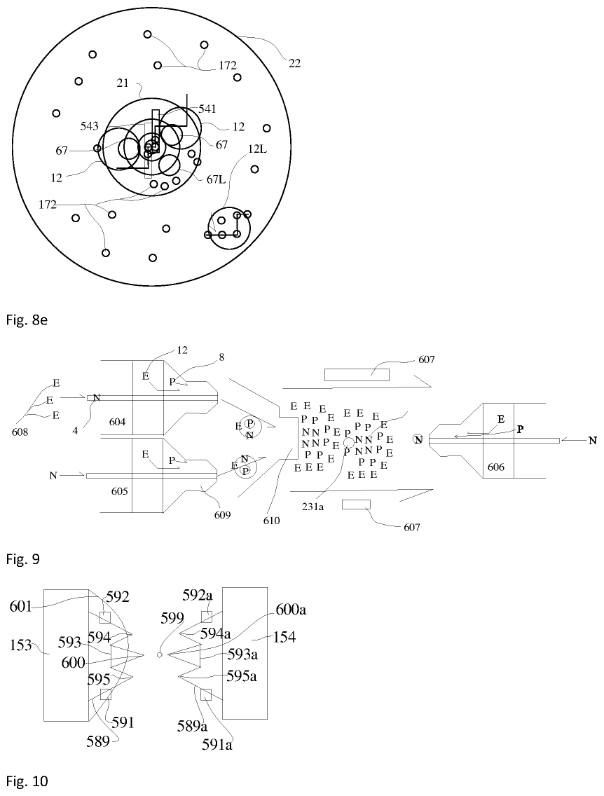

FIG. 8 shows the alternative of a carbon-helium core. This core is also what Silicon would look like expanded in this fashion. There is a first proton core in the center around an inner neutron backbone 15 differentiated from the top and bottom second proton shells 16 within which are the second neutron backbones 17. This is effectively two Carbon atoms on either side of a Helium. This type of core is arguably accurate because it gives similar bonding openings to two carbons which is a result favored by the periodic table and can be carried further out in modeling of all elements.

FIG. 8a shows a neon atom modeled as a unit.

FIG. 8b shows Argon collapsed, while FIG. 8c shows Argon expanded following the model concept of FIG. 8 with ejected protons 620 from the top and bottom proton short neon shells 621 and 622. The competing models both have something to say for them. FIG. 8d shows Helium, showing the Helium shell 555, the single resulting engine 537 and how fractal design can render electrons 12 balanced within shell 130.

FIG. 8e shows a Lithium ion within Li matrix 22 which contains a loose electron 12L essentially dissolved by the free T12 states 172 which balance the ionic proton 67L which would otherwise be bound to electron 12L which are within a second matrix 21 interior to matrix 22. The “space” between the drawn elements is filled with lower ct states which are continuous directly or with elements outside of the matrix 22 with the elements within matrix 22. Unpaired electrons 12L are open when disconnected from the positron of a proton. Separate or dissolved electrons are opened by ct4t11-ct4t12 states, here shown as just t12 states 172 which are comprised of ct4t11 states. The t12 form n is even, for t11 it is odd and the resulting exponent is, respectively, closed (even) or open (odd) mathematically. An electron is “open” since it is 5.4 closed t12 states along an open ct4t13 f-series arm.

a) The loose ct4t12 states 172 shown reflect the amount of free energy in the matrix of this atom. If that is increased, as with the t12 states coming from the solar panel, then the electrons can move from the sharing, closed state to their free open state. This is a break from the “pair of electrons” as a t12 to two 5.4t11 “electron halves.” Two protons 8 are bound by positrons (not shown in this view) to electrons 12, the binding being represented by having item 12 around item 8; one proton 8L is separated by the dissolution from dissolved or free electron 12L. This allows the Lithium to become more “Helium-like” and it can migrate within a semi-conductor. This feature allowing Lithium-Ion batteries charge and discharge can be targeted using different solutions of lower ct4t11 states to maximize the effects and efficiencies of Li or other component motion and organization.

4. Energy

Energy manipulation may be dimensional manipulation through releasing or capturing high pretime change (HPC) ct states within more compressed ct state matrix especially at the photon-electron interface.

Energy is the release of changing ct4t11-12 type information from a higher dimensional matrix and work is using pre time change in conjunction with lower or non-pretime change ct states during the same time frame for relative work/results. Modeling suggests energy is the capture and release of ct4t11 particles (think photon building blocks) in molecular matrix based on the amount of pretime change in specific ct4t11 particles (energy).

Releasing or capturing includes targeting at least two different ct state matrix aligned with KFE to maximize the capture or release of specific ct states within at least one of the at least two matrices.

Storage of energy involves concentrating pre-time elements or change pretime change ct states within a ct state matrix.

It is just as likely as not that by a f-series fractal amount the number of electrons is different, in fact by a factor suggested by the relative size, proportionately less than protons just as protons drop from neutrons.

Charge issues only are experienced by the proton openings in the outer portions of atoms as described here.

5. Fusion:

Fusion can be accomplished by 1) balancing a neutron backbone about at least one AuT fulcrum stabilized by a core of protons and/or 2) balancing a ring of ct4t15 states about at least one AuT fulcrum to collapse a proton into a neutron.

These are respectively Neutron fusion and Proton to Neutron fusion.

The elements being balance about at least one AuT fulcrum, a fulcrum at least partially of lower ct states about which higher ct states balance, involving overlap and folding for stability. The elements of stability are critical for applications of AuT, fractal alignment on either side of the fulcrum, folding around the fulcrum in the form of balancing absorption and spew.

Neutron backbones form a foundation for proton cores which stabilize the neutrons by providing information necessary for the neutron to continually “absorb and spew” information.

Proton to Neutron fusion requires ct4t15/ct4t12 folding around a stable ct15 backbone with supporting ct states along overlapping fractal lines absorbing and spewing to maintain the collapse of the ct4t12 states in pairs within the ct4t15 states paired as shown.

The process includes using exchange of ct state features between at least two matrices to remove non-compressive ct states, increasing compressive absorption in ct states, balancing ct4 states within the neutron backbone with ct4 transitional states, overlapping core ct4t15, balancing absorption and spew at each stage as well as the net release of excess spew in the form of decompression pretime information.

The ct state matrix (typically referred to by crude terms like atoms or combinations of atoms) can be imbued with compression tending KFE features, crudely referred to as being sequentially energized into plasma and de-energized (collapsed) about neutrons accelerated by lasers, magnets, mechanical compression, or other compression means preferably along KFE mandated fractal lines to ensure a steadied result.

An example of generating plasma around neutrons is with microwaves through a conductor, contacting at the points of the conductor where balancing plasma is desired around an overlapping neutron core. This means pretime states at microwave pretime changes excite the separation of protons and electrons from metals.

Opposing like magnetic fields is a new source of AuT plasma at the energy level, generating space through either a fusion or fission type reaction at scale to create or release space. This is the source of magnetic repulsion.

FIG. 9 shows Top plasma canon 604, bottom plasma canon 605 and offset facing plasma canon 606 placed using KFE to place protons 4, electrons 12 and neutrons 4 in a shaping chamber 610 and between shaping magnets 607 to generate a structure and stabilize plasma at different ct state levels. The example here is for fusion, pushing neutrons together with balancing plasmas 231a paired and structured to encourage the type of bonding taught with KFE. Under AuT Neutrons can be directly pushed through their unseen absorption and spew, but for clarity, pushing charge states 608 are shown used behind neutrons which can be accelerated by means known in the art to push the neutrons into place.

Shaping exits and offsets as shown by the offset facing plasma canon 606 are used together not to stabilize plasma, but to place the elements of a stable atom together so that the elements can fuse. While shown for neutron fusion, the same process can be seen to cover any type of compression with fractal equivalent units as will be better understood with the additional disclosure.

Neutrons 4 can be used to draw in plasma, electrons and protons and this modeling of absorption, backbones and cores can be targeted to create the balanced matrix and folding along fractal lines to get internal aligned neutrons 611.

Multiple and successive streams of ct states of a certain compression state can stabilize one another. Electrons can be concentrated and mixed with protons and then a central neutron core is added as the charged particles are concentrated and accelerated out to stabilize them. The protons central to the electrons but having them start on the outside might be beneficial as they would pass through the ct4t11 clouds pulling everything together. Likewise, having a negative charge to the neutron tube especially towards the end might help to pull protons towards the neutrons which can be accelerated by having them pulled along by a strong electromagnetic accelerator and the separately accelerated protons (opposite charge) due to the shared absorption from the protons as the EM field is reduced.

Projected fusion is where the elements are pushed together, shaped fusion is where the elements are in a cage which encourages stabilized alignment of the elements.

The protons (P) can also be provided in the form of metallic generated plasma from microwave level energies through conducting wires making contact within a mix of heavy hydrogen which would provide neutrons.

AuT Plasma canons can use mechanical (shaped as by funneling or chamber shape), electromechanical and sub-electromechanical (below ct4t11) means to concentrate the balanced matrix and organize multiple AuT plasmas at different levels. Facing plasma streams as shown particularly with an offset can encourage rotational balance, with different canon supplying (1) AuT plasmas or each stage of fusion and (2) stabilized fusion ct states or states to balance the fusion ct states. Here there are two cannon systems having at least one offset facing another, to achieve fractal balance and to increase the pressure and potential for fusion within this model.

The number and arrangement of the canon can be determined both by the ct states and the order in which they need to be added, the need to maintain the neutron backbone and plasma between neutrons in place and the fractal shape of the resulting atoms in addition to other KFE which includes the relative energies necessary to bring together the ct states to be fused. Directed energy” or “plasma ignition” is analogous to concentrating states with high pre-time change rates (tending towards gamma rays) from those with less pretime change (tending towards infrared).

KFE includes Neutron donors within the plasma distributed to encourage overlapping neutrons with surrounding protons and electrons on either side of stabilizing AuT plasma fulcrums discussed below.

To bind plasma or get fusion consider a neutron absorbing (Neutron short atom, short of a neutron to be balanced) or neutron granting (such as deuterium which has a loose or unbalanced neutron) in the middle or from either side. Removing non-compressive and/or changing ct states is also envisioned. Plasma represents dynamic balancing of information, typically using rotation for balancing of strong electromagnetic abs and spew. Plasma may be delivered and triggered by having it trapped with higher ct compression states which are removed quickly to allow the plasma to be delivered at the points necessary to provide AuT fulcrums.

6. Catalyst

Catalyst action is maximizing interactions based on Designing around temporary transitions within a matrix between low and high pretime change giving rise to ct, state sharing effects at higher compression states relative to at least one second matrix which is targeted to be affected by having these temporary ct state exchange locations.

This can be done by creating and Targeting shifting AuT fractal hinge plasma centers targeting the migration of information between plasma centers within a matrix and expansion or contraction of the plasma centers with a matrix to increase or decrease the ease of migration of information.

Shifting plasma cores within ct states; move/expanding/contracting (change relative size) core 1 to do work then core and where it is a Catalyst reaction plasma shift and using heat to expand plasma to allow shift to ease moving in and out of balance.

New designs of rare earths (RE)show that they are modeled around an expanded Neutron to Neutron plasma fulcrum. One improvement is to target this new design, as with other chemistry, to get better catalytic effects, not just of RE but also in all catalytic chemistry. AuT redefined the atom, showing that atoms are bound by absorption of information by the neutron backbone which is stabilized by the proton core around it which is, in turn stabilized by electrons which are halved forming pairs along the same overlapping spiral either with positrons extending from the protons or by associated, bound atoms. Catalysts can be formed and their performance maximized by focusing on the expansion and contraction of at least. AuT fulcrums within the catalyst so that as one is expanded, the information is drawn from the other contracting it and when balance is returned, the fulcrums, having performed their work in the expanded or contracted form (or both) has been returned to a balanced state. The discussion on Rare Earth elements shows how the AuT fulcrums are expanded in those multi-arm atomic states.

AuT allows targeting of low energy plasma centers in atoms and molecules to allow simplified or at least modified manipulation. This process is to redesign targeted chemical reactions to increase separation efficiency. Picking one element or source is contraindicated in this modeling since it can be applied to any material and should be broadly applied for purposes of maximizing mineral extraction; but for purposes of this application, the example given is in rare earth elements.

7. Electricity, Energy, Plasma Centers and Empty Space

To maximize the return of a radiation (e.g. solar panel), KFE features of the wave and absorbing atoms can be matched for easier information sharing and targeted ct states may be drawn out from the absorbing atoms to draw in the targeted ct states from radiation. Prior art defines “charge” deferring to how it behaves and quantifying it in terms of what is measured noting the smallest units of charge are the same size. AuT shows that energy is pretime change in ct4t11 type pretime states. Photons appear to be ct4t11 composites and the generation of energy is the release and concentration of these states with ct4t12 based on the pretime change in the ct4t11 states. Electrical transmission lines operation, by way of example, can be modified using high pretime change for greater efficiency using KFE. to ct4t11 type states held in proximity by the absorption and spew of information between the electrons in the wires and the surrounding ct4t11 “stabilizing field,” in AuT “the matrix of ct411 and lower compression states surrounding and stabilizing the higher compression states in the wire.

While Electrons themselves are relatively bulky, the electron components ct4t12 states are not as much and the extent to which the actual movement of charge is in the form of the 5.4 ct4t12 state electrons freed from positrons extending from ct4t15 states (or a matrix of ct4t15 states) dissolved with higher levels of ct4t11.

Electrical transmission is well understood up to the level of fields. Those fields are addressed here. In AC circuits, electrons do not move over long distance, but the fields move at the speed of light delivering energy at great distances. The prior art provides differing interpretations, AuT modeling explains the process using fractal mathematics to allow designing components at all levels increasing efficiencies.

In grid systems this is transferred to surrounding fields through the absorption and spew process by which ct4t11 states surrounding the wires exchange ct410 information with the electron shell around proton cores around neutron backbones in atoms.

Insulators contain the information within the electron shell drawing in the “free ct4t11” states as they need to get closer to exchange ct4t10 information with the electron shells which are contained by insulation and pressurized by the addition of energy which would otherwise expand those shells outward.

a) Protective relaying: Understanding the process by which electricity is transferred and allows for the transitions to be ameliorated, whether in spikes or shorts or otherwise and for better prediction of how these transitions occur.

b) Predicting performance Understanding the process by which electricity transitions between different electronic components allows for the performance of those in different situations to be determined and improved.

Conductivity is misunderstood because the nature of energy is misunderstood. Indeed, atomic structure is inaccurately portrayed by leading models of quantum science.

AuT defines the operation generally of the neutron backbone of atoms and the function of absorption and spew of lower information states which not only gives rise to the observed forces through folding and unfolding of different compression states; but also the exchanges that give rise to conductivity and the new atomic structures which do not rely on electron orbitals for the resulting features of atoms, at least not in the traditional sense.

1) There is a shift in the field (spew) through which the solar system is moving. Since this is a rapid transition, it means the absorption and spew lines are near adjacent. In a fractal universe (and the denominator of pi and pervasive curvature are compelling evidence) you have this same phenomenon at all levels.

8. Electromagnetism, and Pretime Poynting Vectors

ELECTROMAGNATISM (EM) is defined by both the direction of the flow of the ct4t11 and likely ct412 states dissolved in a ct4t11 matrix. The suggestion of vectors is that magnetism represents the movement of a ct4t10 toward a ct4t11 state and electricity is the movement of ct4t11 states towards ct4t12 states.

Electromagnetism involves change in the ct4t11-12 range. Lower ct state levels cause the Poynting Vectors necessary to have the parallel circular field of magnetism as opposed to the straight flow down a funnel you would observe with a fluid where the external ct4t11 merely flowed into the wire and then down in the direction of the current flow. Each pull inward requires lower ct states to move outward so that the observed movement is a counter-intuitive function of compression or decompression.

The process can be thought of as applying KFE elements to design radiation matrices for electronics, solar, thermal and radioactive energy use, capture and dispersal.

EXCHANGE of lower ct states in a higher ct state between AuT matrices is interaction. If the particle, for example, was to exchange just the 10 changing ct states at 90 degrees with another ct4t11, then that ct4t11 would tend to list in that direction (the 90 degrees to the first) at that much greater of a velocity and tendency, with that much more force in that direction and other ct4t11 states along that line might pull in other directions and the difference in pull from those states exchanged. In arriving at a net change in the two matrices, there is no contact, only exchange of information and by exchanging these lines, the direction of the two particles change. If, by way of example, item 550 was given to another particle, then that particle would be biased in the direction of unwinding of 550 relative to the matrix in question.

A narrow example is using KFE features to have EM and insulation work maximized by keeping the ct4t12 states within the electron matrices and forcing ct4t11 states surrounding and stabilizing the ct4t12 states to remain closer so that absorption and spew can be maintained, the insulators themselves being porous to the ct4t11 states.

KFE underlying Poynting manipulation can be used to modify frequency-based systems increasing the efficiency of those systems for generation of energy, transmission of energy, use of energy and storage of energy as well as increasing the overall accuracy of results.

KFE is information exchange instead of collision in absorption and emission of em radiation in electrical components and field modeling.

The flow of energy goes from low to high pretime change. It is either lost to surrounding matrix or moves in whatever direction is available channeled where it shares information in the direction of winding or unwinding about KFE of ct states.

Poynting vectors are defined as: S=1/u(o)(E×B) or S=E×H where e=electric field, B=magnetic field are useful in understanding what is happening in the model. Charge changes the direction ct4t11 is biased, as charge moves the change in this pointing and the associated movement is magnetic. The surrounding ct4t11 about a wire is drawn in at points where the charge moves inward. As ct4t11 or ct411 composites (made of multiple ct4t11 states), possibly as ct4t12, is pulled forward in wires, it has to be replaced with ct4t11, going into the wires. What is pulled from one side of the Poynting vector is pushed out by the other to maintain the balance, which is imperfect, but in an EM circuit it occurs in sufficient balance that spiral outward and inward states yield the observed curved fields around wires where ct4t11 states are pulled out of the wire matrix (negative) and the side of the wire matrix where they are pulled in the positive side.

Folding of information or unfolding into compressed or uncompressed states below the level of the ct4t12 component electron is pretime, at the al level information goes in different directions and completely in and out of dimensional perspectives. At the ct4t11-12 range, these differences are between electricity and magnetism,

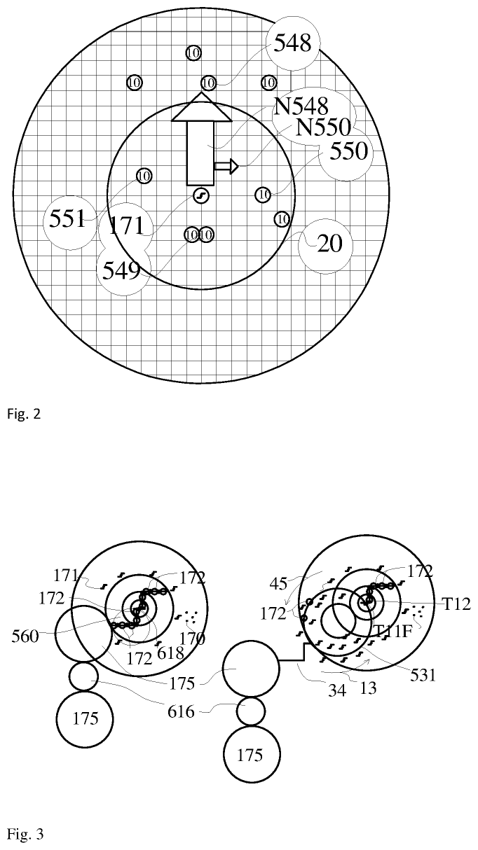

FIG. 2 shows how information transitions between electrical and magnetic features with pull in each direction, along the path of 3 dimensional travel, here indicated by the “net unwinding” Net information exchange 548 made up of decompressing 548 and compressing 549 through movement of ct4t11 171 relative to the first matrix 20; and magnetic elements made up of the net magnetic decompression N550 made up of the magnetic decompression 550 and magnetic compression 551 must be the same over time.

In fact, if the t11 state is to remain stable the amount of information going into it and that going out must, over periods of absolute change stay relatively the same. They are not the same, however, because quantum fuses for points are different and that type of consistency would be an aberration.

The effective size and direction of the transitioning ct4t10 states is shown acting on a single ct4t11. A ct4t11 is 10 ct4t10 states, a ct4t12 is 10 ct4t11 states and an electron is effectively 5.4 ct4t12 states, an electron pair 10ct4t12 states, effectively a ct4t13 states.

Side pull can be viewed as magnetic effects, net straight-line as electrical effects. Side effects can be artificially increased.

Pull of ct states from a matrix are spew, and if the matrix is maintained it is matched with absorption, both along spiral lines giving rise to observed magnetic fields.

While limited to the size of the matrix shown, for different ct state folding defined forces, the results can be treated the same way as for magnetism for any given area, the greater the matrix size, the smaller the effect of the changes of any ct state within the matrix.

A ct4t11 photonic state is shown in the center. Area of overlap at the center of this ct4t11 state is given a value of 1 and the circle reflects an effective rate of change 250 times the size of the overlap, the pretime change potential.

The energy of a ct4t11 remains the same for longer than quantum changes because the pretime elements folded together to make those ct states change sequentially and in a pretime environment. The pattern is fractal and therefore self-replicating and at ct1 each change is sequential from one point to the next, but fuse length changes complicate this process for folded particles to give observed results at any point in the universe rendered largely consistent by averaging long fuse lengths at our location and stage of the universe. Change once begun is continuous from one point to the next, but separated by fuse length which at the ct1-ct3 is time independent, but netted out positive or negative results appearing together from a time based perspective.

The larger part of this pretime change is in one direction and external to the particle, it is the winding and at this stage of the universe primarily the unwinding which means that at the core of compression (ct1) one particle after another is changing state relative to the net fuse changes towards compression or decompression across vast distances of the universe.

The relationship between absolute change and dimension is reflected in fuse length which changes for each point for each quantum change; but pretime change is a subset of this which reflects compressive and decompressive changes at different compression states which are perceived as force. The relationship between plank length distance and quantum change is reflected in the ratio of the speed of light to gravity which is the ratio of electromagnetic transitions at ct4t11 to quantum dimensional change (gravity).

While shown as linear, folding and unfolding dimensionally is along recognizable fractal lines, f-series spirals. These information streams are only linear from the perspective of electricity concentrated as with straight wires and magnetism reflects the surrounding nonlinear movement (linear being the line of electrical flow) as ct4t11 states spiral down to the ct4 state shown in the center. Replacement is inherent in a circuit. Since the particle matrix 20 remains essentially the same, the H and E of a particle appear to remain very similar, especially when averaged over time.

The pulling of a wire through a coil (generating electricity, whether through movement of the coil or wire) can be seen much like attaching cotton candy to a paper funnel except more continuously, as the pretime information added is drawn along and other information around the coil is drawn in to replace it.

Magnetism reflects the exchange of information as rotational absorption and spew, such as is shown in plasma and when that goes linear it is electricity. Magnetism appears due to attraction along the line towards (or away from) compression, whereas electricity is movement parallel to this line of compression. The line of travel is electrical and the surrounding pretime transitions reflected in a net result outward or inward are the magnetism component. In a balanced particle, the inward and outward pull net out the same giving the spiraled pattern seen in magnetism and the rotational movement (Archimedes wheel) of the waves along these lower dimensional to higher dimensional spiral movements when viewed from the post time perspective as a wave.

Since the relationship between electricity and magnetism is determined by an offset of 90 degrees, AuT suggests this is a half fold is 90 degrees and this suggests that magnetism states are ½ of a fold towards the next state which for nomenclature purposes can be a ct11:ct12, a ct12 to a ct13; etc.

While it appears that 5 ct4t12 states make ½ of an electron pair and while photons may well be 5 or 10 ct4t11 states without more study, the 90 degree offset, electron component and photon states are designated with ct state designations as the ratio of ct4t11:ct4t12; ct4t12 and ct4t41 states respectively understanding that with additional experimentation these designations may be adapted as long as the basic fractal concepts are maintained, being modifications of details unique to AuT.

Movement causes EM field by interacting with the surrounding stabilizing field (ct4t11 states being impacted by the moving ct4t11 states carried along with the ct4t12 states which are moving through the wire) which change at higher pretime speeds than the underlying electrons allowing more or less instantaneous transitions down the length of the wire and creating the effect of magnetism as other ct4t11 states are drawn in or pushed out in a pretime environment to balance the disrupted matrices.

Changing ct4t10 states in the single photonic ct4t11 is simultaneously going on for larger and smaller ct states across vast distances and defined by the size of the matrix, here shown as the relative informational size of a ct4t12 state with a ct4t11 in the middle; but this modeling applies to all fractal states, the relativistic observation coming from the time/pre-time observational perspective, false relativity also called relativity.

The average “change” trapped within atoms and molecules in in folds, often with considerable tension as with fissionable materials, is similar because of averages in largely stable, but releasable forms. These can be transferred and focused so that the energy from heat is focused just as is done with other forms of energy and this can be extracted from any ct state form it being understood that the size of the ct state in question is less relevant than the amount of pretime change of the lower ct states making up the ct state in question. Transferring the pretime change states efficiently can include having the photon lowered in energy as the pretime changing ct states in the photon are exchanged, shared or transferred to an electron utilizing KFE.

The more of these pretime changes, the shorter the wavelength. Since they are quantized, they reflect the quantized size of wavelength changes and since they effect different ct states, the changes to those ct states are also quantized giving rise to increases in orbital energies of electrons as that pretime information is transferred to electrons and their associated clouds of lower information states.

Under AuT electrons can migrate using electromagnetics tied to the constitute t12 states and t11 sub-constituent states. The ct4t12 states can move in a “solution,” dissolved by an excess of ct4t11 states so the entire electron need not move, ct4t12 states can move alone. Waves are not absorbed, so much as constituent parts are spread out in the electron portion of the atom matrix.

9: Collisions as Information Exchange

KFE includes targeting fulcrums and stepped transitions. A t12 state shares information as a unit of a t13 state and is composed of lower states which can exchange. The pretime change in the moving t11 photon exchanges pretime change sequentially with pretime change in multiple other t11 states substitute or enter orbit with other ct4t11 states within the electron. Enough pretime change can be transferred in the form of exchanged or contributed ct4t11 states to the electron to move a photon out of orbit which orbit would be a matrix of the type shown in FIG. 2. Since all ct states are quantum, orbital changes related to absorption of et states are quantum, tied to how much exchange of information occurs in the matrix in question.

Speed of light and Planck length are eliminated and pretime changes at lower levels are averaged. Quantum change (x) begins as a very large number, scaled with an effective rate at a scale like 10\hat{ }44 changes per second. Ct state (not time) change (dimensional change) is a time alternative so that in this example of time vs. pretime change 10\hat{ }44 is replaced with 1 at the point of post time observation, and x=10 equates the area occupied by the ct4t10 states making up the ct4t11 with the ct4t11, x=100 makes the ct4t10 equal to the ct4t12, and so on. These are discrete changes in size but they can be broken down by ct state for energy based phenomena or other KFE features which result from lower ct changes within ct state matrices; in this case between the ct4t11 and ct4t12 states where movement of photons and electrons are detected.

Energy is the effect of the amount of space of a ct4t11 state taken up within the electron when viewed from the perspective of time and this space is taken up by the exchange of information related to the ct4t11 within the electron.

Collision is a function of angle at post time scales. Angle is equivalent to the amount of information exchanged, a direct hit is a zero exchange with a reversal of direction of the contacting particle being a near equivalent measure, any other exchange of information varying the effective angle, a 100% change being a transfer of changing ct states within the ct4t11.

At no speeds occurring in nature do neutrons from high energy sources combine with neutrons of atom with direct sharing unless there are fusion or near fusion information sharing and those do not remain stable without balancing. There are no collisions per se, but there is sufficient sharing of information to create collusive effects when viewed using pre-AuT science.

The high energy neutron (HEN) example is instructive, high atomic number and low atomic number and neutron absorbing materials. This is done with Lead, (Id and H and B. In a fractal universe, this model can be used for radiation, merely lowering the fractal modeling from ct4 (ct4t16) down to ct4t11, Lowering a HEN to a thermal neutron is lowering the pretime change ct4t11 states within the HEN matrix from radioactive levels to thermal levels and as the information is dispersed, more is shared in the proton-electron sharing range leading to increased vibration (thermal).

10. EM-Plasma Extension and Magnetism Creation at Higher Energies

If one looks at how to use repulsion in fusion (or other reactions) there are two aspects, one to push together reactants and the other is to draw out vacuum. FIG. 10 shows inner magnet 153 and outer magnet 154 and between them is what AuT predicts is a repulsive flame 599. The view of iron filings is instructive in repulsive magnets. The iron filings form a bulge 601 towards the center, falling away to either side of the magnets, here shown for magnet 153. This bulge 601 looks attractive and the explanation is likely that information is being drawn in (absorption) to the repulsive flame 599 (generation of a spatial ct state/repulsion) between the magnets 153 and 154 which flame 599 generates spatial features which push back flattening out the iron filings toward the edges. This means the flame 599 is a repulsive center which is also an absorption center which can be steered to manipulate ct states.

To steer the location of flame 599 the bulge 601 is replaced with guiding left guiding wire 590 facing 590a to guide the draw of ct4t11 states from different directions to change the location of the flame 599. Multiple points of maximum extensions 594, 595 and 600 of the guiding wire 589 matching extensions 594a, 595a, and 600a of wire 589a to manipulate the feed of ct4t11 states toward flame 599 which are controlled by circuit breakers 591, 592 and 593 in wire 589 and breakers 591a, 592a and 593a in wire 589a to change the effects of these transitions.

If one of the magnets is replaced with a wire or if the flame is replaced with a wire and the remaining magnet(s) are rotated the movement of the point of compression creates an AC current and rotating magnetic fields which can be rendered more efficient using the process disclosed herein of using the KFE involved to maximize the efficiencies of the pretime change transfers regardless of the number of phases. These efficiencies can be practiced in reverse as with an electric to mechanical transition (electric motor) where the same modeling and resulting efficiencies can be brought to bear, as by targeting ct state movements around and between each component, typically the three phase wires (of the stator), rotor components, particularly focused on iron (conductors) and commutator.

FIG. 3 shows traditional plasma 531 (as opposed to AuT plasma which refers to a broader group of fulcrums). The proton-electron interface is effectively a positron 560 extending from a proton block of 10 t15 states 175 (only two are shown here) connected to 5 ct4t12 sates 172 which form an electron shown bridged on the left by positron to electron AuT plasma fulcrum 618. When excited by pretime change, fulcrum 618 expands into traditional plasma 531 which is still a fulcrum. AuT plasma fulcrum 616 is between the ct4t15 states 175. Additional ct4t11-12 states with more pretime change within the matrix of 5.4ct4t12 cause the expansion.

Plasma is the effect of separating the proton-electron interface here with ct4t10, t11 and t12 states with absorption (towards the proton) 45 and spew towards the electron 13 within a fractally significant, therefore stable plasma 531 here equal to the informational size of the proton. Plasma 531 is itself a matrix like Matrix 20 in FIG. 2.

The free ct4t10 and t11 from a post time perspective appear larger than they are because of the number, positional change in pretime states and exchange information in enough locations of the plasma to expand it, and enough information on either side (proton and electron) to keep the plasma 531 separation stable.

Electromagnetic properties can be targeted by the fractal size and amount of pretime change necessary for balancing and modifying balance focusing on the alignment and structure at any level. Interaction between layers at the atomic-molecular level focus on the strength of the overlap: Fusion is neutron to neutron information sharing; 2) Molecular is proton to proton sharing marked by; 3)Stabilization of molecular is electron to electron information sharing based on the amount of sharing at the proton level because the electrons that are shared, have to share information with exposed areas between protons to get molecular bonds as opposed to electrostatic bonds which would involve net charge areas and not true bonding sites. Pretime change is the equivalent of movement so what is being transferred is the amount of movement, ultimately pretime change, over relevant sized quantum changes (ct4t11-12 in the case of electromagnetism) which is dispersed into a larger body through interaction at multiple locations at the proper fractal location using KFE.

Before AuT radiation was averaged and treated as field effects and electromagnetic (em) waves did not require a medium to propagate. Under AUT, EM waves are fractal ct4t11 states moving pre-time (ct4t11 being photonic building blocks of 5-10 ct4t11 state photons) which propagate in a medium of pre-time fractal states smaller and less dimensional than ct4t11 photonic ct4 states referred to generically as space. EM waves are created due to moving of electric charges, but this is just another way of saying that ct4t11 states which are largely pre-time and photonic in nature, currently viewed generically and incorrectly as moving within vacuum, and ct4t11 states are freed and accelerated through these lower ct states, perceived as space, where their pre-time changes appear as oscillations of electric and magnetic “field.” They can no more exist in isolation than a neutron as all ct states require balancing of absorption and spew of information to maintain their dimensional stability due to unfolding although isolation and stability is possible because of long fuse lengths.

The electric field in an electromagnetic wave vibrates with its vectorial force growing stronger and then weaker, pointing in one direction, and then in the other, alternating in a sinusoidal pattern in response to absorption and spew variations. At the same frequency, magnetic fields oscillate orthogonal to electric field. The direction of propagation of the wave is perpendicular to both electric and magnetic fields and reflect rotational changes. The pre-time effects, absorption and spew requirements and locational features can be targeted for purposes of energy absorption, redirection and shielding.

Electrical (along a line) or magnetic (rotation outside or perpendicular to this line) views of electromagnetism reflect information unfolding (from ct4t12 electrons to ct4t11 photons) along a line of compression towards or away from the next higher state, namely the electron (5-10 ct4t12 states, 5 ct4t12 states being an electron stabilized by a proton-based (or at least proton area based) positron and 10 ct4t12 states being an electron pair free of a proton-based positron.

Absorption 45 and spew 13 give rise to conceptions like angular momentum in an electron, pretime information exchange, only complicated by relativistic effects which are illusory, the illusion between net pre-time change and incremental post-time change. One can see even at the level of FIG. 3 at the proton/electron interface, the “matrix” is complicated by multiple pretime states and as shown in FIG. 2 by states which are connected to states within the matrix even though they are exterior to it. Hence the term “AuT Matrix” defines those states which are artificially isolated from the rest of dimension so that impacts due to changes in ct state sharing can be manipulated.

11. AuT Fulcrums

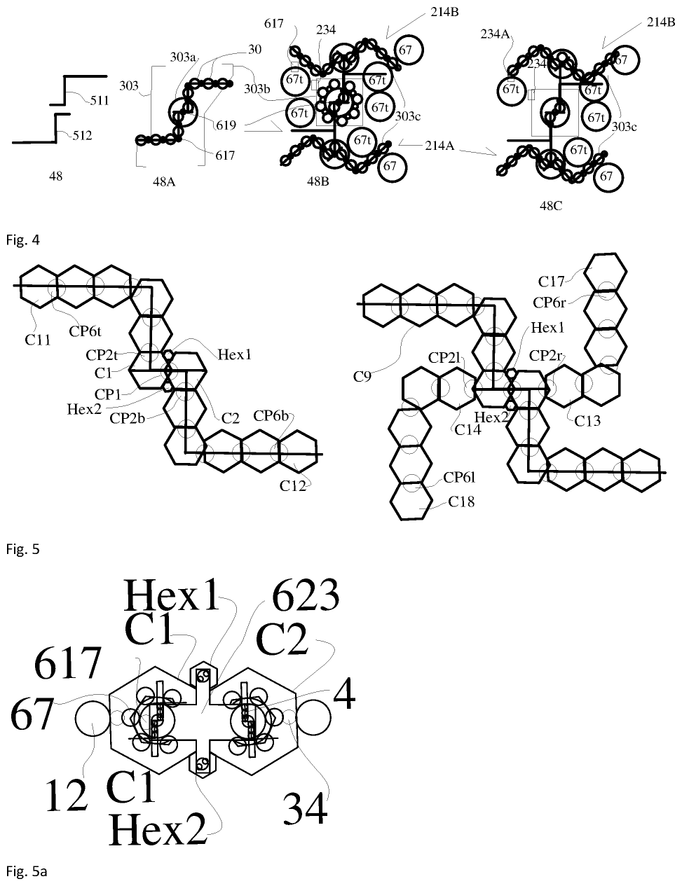

FIG. 4 shows a broader view of AuT plasma fulcrums about which higher states balance, here aut plasma fulcrum 619 is the central most neutron to neutron plasma fulcrum, everything else balanced and folding around the fulcrum; balanced by absorption and spew between the elements. Added or inherent to these, is the fractal form in balance (the linear spirals), plasma centers between ct states, and net absorption leading to the folding. A fulcrum of lower states about which higher states balance, fractal alignment on either side of the fulcrum occurs because higher ct states form within shells of folding lower states, here fulcrum 231 within combined neutron fulcrum 303a is the central neutron pair supporting neutron spirals 511 and 512 which can hold surrounding combined neutrons. Balance and folding around the fulcrum is by absorption and spew between the neutrons. Internal to this is a Neutron to Neutron plasma 617 for neutrons 30 outside this core. Neutron to proton AuT plasma fulcrums 617 are present as absorption and spew to balance that connection. Inherent is balance of linear spirals 511 and 512, net absorption leading to the folding or closing in of the spirals around the areas of overlap represented by fulcrums which incrementally increases the compression according to the observed rule of 2f(n)\hat{ }2\hat{ }n. Imbalance using lower information states are used to capture or change force features. The direction and form of winding and unwinding into circles or spirals and can be used to maximize ct state exchanges to transfer information and maximize work.

Doing work with sub-electromagnetic ct states is new to AuT because these were not recognized before. This is important for quantum computing but can be used for a variety of purposes. The best example of doing this with ct states has to do with Magnetic fields reduced to ct state effects. The attraction of like changes is folding together of ct4t12 states from positrons to electrons and is electrical. But what of the repulsion of like states. AuT cannot just rely on banalities such as “opposites attract and like charges repel.” There must be a ready explanation between absorption and spew (compression and decompression). The most likely explanation initially appears counter intuitive. Features of “aligned” ct4t11 states coming off of ct4t12 states “fuse,” and when they do, they release lower ct states from the pretime ct4t12 states similar to fusion, this released information pushes things apart in a mechanism similar to spatial expansion so that its effects are primarily felt between the “field” states of ct4t11 giving the effect of repulsion matching the folding together in attractive magnetism and electricity.

Aligned outer spirals 303c are shown in FIG. 48 c which can spiral and fold, while 48b shows an arrangement where the outer spirals 303c are not aligned and cannot fold inward. The force of folding in magnetic fields is not observed explosively because it is small and pretime. This leads to opposite states which are decompressive from a positron and compressive to an electron so the openings in positrons draw in those decompressive from the electron and combination occurs, but closer to atoms and there is a net absorption of space, at least the dimensional repulsive kind. This is not counter-intuitive from the standpoint of spiral compression, because the alignment of “like states” for overlapping compression is easier to envision. Galactic spirals must be aligned to get folding, the same is true at all fractally similar levels. The positive and negative versions of the ct state in question can fold together with the underlying absorption or spew positron or electron source.

Overlapping spiral forms suggests between each arm and each element there should be half as much shared information dimensionally when perfectly balanced. Here the overlap is 2, 4, 10 with each half being 1, 2, 5 The shared information is not “space” since all dimensional features are some forms of ct state, absorption, and spew ct state sharing. Treating fractal representations 48A, 48B and 48C (which could be fractal equivalents of photons, electron, protons, or atoms) together as atoms; there is a neutron backbone 303.

External protons 67 are differentiated by trapped protons 67t within the outer neutron backbone formed by bottom overlapping neutron spirals 214a and top overlapping neutron spirals 214b which shows how protons can be mixed with neutrons in the core. The outer spirals 214A and 214B in atom 48C are shown aligned for folding, but as shown in 48B one or the other would have to be mirror imaged to allow the type of alignment seen for a galaxy and to achieve the type of folding shown here for the secondary backbone 303b in atom 48C.

The views 48b and 48c show outer arms 214a and 214b aligned in terms of folding (48c) or unaligned (48b). The unaligned arm 48b might show why Protons fail to collapse or why some states remain decompressed since the top and bottom arms are not aligned.

This modeling is consistent with Xe (77) modeled with 10 carbon elements shown with a 10-unit Ar backbone (equivalent to item 303a, but fractally 5 times as much ct4.

For a simple atom: R (radius of electron orbital=e(o)*(n\hat{ }2*(h/2p)\hat{ }2)/(pi(me\hat{ }4)): e(0)=vacuum permittivity constant; m=mass of electron, e=charge of electron; h=Planck's constant; all but n=Bohr radius. N=quantum number 1, 2, 3, etc a radius yields 2\hat{ }n suggesting 2\hat{ }n yields the electron radius. These numbers begin to fail for multiple electron atoms, this can be understood in terms of the amount of dispersion associated with the new arrangements of the areas in which particle are expanded in a fractal atom and the resulting dissociation from the single positron/electron interface where the change in the proton absorbed is matched with the change that an at rest electron would have which is where the AuT antennae function as matching the length of ct states within the AuT antennae with fractal elements in absorbed ct states to improve antennae function at different scales by focusing on this relationship for all targeted ct states.

Bonding is made stronger by focusing on neutron-to-neutron bonding and resulting areas between protons rather than electron bonding. The method involves maximizing desired neutron balancing and unbalancing to stabilize or destabilize desired proton area sharing with fractal folding to pull the ct states together about the various AuT fulcrums between ct states at different compression levels.

The diameter at different levels of pretime change and the amount of change during a given period are closely related, one increasing the height of the waveform, the other decreasing the wavelength. The former operates as an antenna for photon-electron interaction size allowing for the quantum absorption and spew (excitement and discharge) reflected by ct state exchange at different levels.

If the average change in the ct4t12 state is equal to the much more information dense ct4t13, paired electrons, it would have an effective change size equal to the electron from a post time view. The shared information occupies a second orbital defined by the 5.4 ct4t15 states plus the ct4t12 state. Absorption represents the shared ct4t12 information in the ct4t13 matrix and the energy drops expelling the photon collection of ct4t11 states, some of that exchange remains or is exchanged with the ct4t13 matrix.

KFE can be used creating ct4t11 core antennae which can pull the radiation from the circuits and utilize it for other purposes.

12. Electrical Insulation and AuT Antennae:

The process of claim 1 further comprising applying fractal features beyond shielding exist in AuT insulators and AuT antennae, for electromagnetic (thermal, solar and radioactive) manipulation (control/capture) through the application of KFE for at least one of AuT antennae and AuT insulators.

Carbon chain Antenna activating p type atomic ct state structures can be used, for example, to replace elements of the ATP cycle. You can reconcile size at post-nano scale sizes, not with actual size, but with fractally consistent spacing at larger scale knowing the components will yield some of those effects at the fractals below those scales. AuT antennae can be embedded to deliver or draw out current.

AuT antennae is information matching which can be done at any scale of compression. AuT insulation is an effect of using a stable high compression, high compression ct state to hold in a lower ct state while allowing the next lower ct state to pass through; in the case of electromagnetics, these are insulators keeping mostly post time ct4t12 states within metal wires while allowing ct4t11 and possibly free ct4t12 states to pass through. The required balancing with pretime states required, pulls the ct4t11 states in closer to give the amount of absorption necessary passing through the insulator to keep the charge maximized while at the same time spew from the ct4t12 states within the wires keeps the external ct4t11 states with high levels of pretime change, providing both the electrical effects and magnetic effects of perpendicular movement ct4t11 states towards or away from the electrical direction along the wire.

Equivalent processes can be used at any state of compression.

Changes in the KFE features of electronics from wire to grids can increase the efficiency of the electronics.

1) Carbon Capture: Insulation concentrating techniques can be used at atomic scales, for example using a CO2 antennae surrounded by sufficiently porous CO2 insulation to pull carbon in so it can be pulled in, concentrated and stored or 2) Charged carbon antennae can be used to draw in radiation, increasing the amount of pretime change which can be captured in conjunction with fulcrum centers to partially capture the rays, slowing them down through the exchange of pretime information at the radiation level.

AuT Antennae at the neutron, proton and electron levels have these different KFE features plasma widths, engine length (lines of neutrons along any fractally significant line), proton and proton shell widths, electron and electron shell widths, positron and positron-electron widths, by way of example.

Shielding with AuT Antennae Type Dispersal Array

AuT matrix can be designed based on the absence of contact which is replaced with information exchange especially regarding pretime change rates in information using KFE. KFE may be applied in fractally relevant quantum steps between the source and target levels. Some KFE features are creating stabilized backbones and cores from destabilized, unbalanced neutron backbone and proton cores. Shifts between plasma centers and back designed as catalyst reactions to allow repetition.

Neutron cages with inadequate proton stabilization can be used to form electromagnetic traps in conjunction with the surrounding protons and electrons. Carbon antennae embedded and activated by drawing out of introducing AuT plasma centers with information donating matrices of atoms.

15. AuT Pretime Change Push Battery Charging

One application of the process of AuT antennae and insulators is in “Fast charging Batteries using AC or DC current incorporating atomic (Activated Carbon Antennae) and electric (ct4t11) concentrating and transmission features to increase charging speed and efficiency.” A side benefit of the AuT model of energy and field charging (adding pretime change to the ct4t11 cloud around atoms) is that it can be used to improve battery design and lower the charging time by using the same techniques to add and remove charge and to make it easier to absorb charge, to distribute charge, to limit dendrite formation and introducing ct4t12 and lower et states to break up dendrites and limiting their formation through the circulation of the ions.

FRACTAL concentration of the antennae arranged as the leads are approached, CHARGING AND DISCHARGING by movement of ct4t11 states or infusing higher compression states with more ct4t11 (possibly as ct4t12) using alternate current; positive one way, negative another way of spiral compression at different stages of compression and to distribute most efficiently the ions moved to charge moving the converter (ac to dc) into the battery itself. The same processes can be used, organizing the pretime change within a battery at different times in the charging process to increase the efficiency of charge and discharge.

Pre-time change and ct structure can be matched to maximize results based on AuT modeling. Flooding matrix with ct4t10/ct4t11/ct412 states to create a better solution, circulation of subatomic and atomic elements of the solution and ions within the solution with biasing towards anode and cathode by sequential charging, relative amounts of charge, and layouts of dissolving ct4t11 state features using KFE.

Battery charge is performed by using AuT Antennae in the battery matrix, here shown with “antennae sized (based on the antennae main trunk or branch sizes and pretime features) ct4t11 states, energizing the electrons, dissolving, usually Lithium, atoms by creating a proton-electron plasma (PEP), a version of AuT plasma, before moving them with charge, and dispersing the same; the target is to increase the consistency of PEP and encourage distribution evenly around the Anode or Cathode. AuT insulating features can be used to gather ions and charge where they will do the best and the least harm around the anode and cathode.

The ct4t11 migrates into the higher ct state matrix which expands and is released outside of the insulation.

F-series Spiral and concentration according to KFE features, particularly the placement and/or size of carbon antennae around anode in one direction, cathode in the other to energize their flow, internal perpendicular to increase that flow. AuT antennae (such as Carbon atom chains doped to increase conductivity), can be insulated and used as on/off cathodes to increase the ct4t11 states within battery solvent and maintain the dissolution of the outer electrons of Li (or Fe) and encourage flow and distribution of the larger ions to allow them to migrate more easily towards metal anode and cathodes.

The carbon antennae charge at the dendrite formation area can be used to help dissolve dendrites through distribution of the ions through changing the flow of ct states during the charge process, control (limit) their formation in the first place by providing a consistent fluidity and circulation of ct4t10-11 states around the anode to which the positively charged atoms are attracted.

16. Conductivity of Metals

An odd thing about wire is that it limits the amount of electrical CURRENT, but it doesn't care much about the VOLTAGE level. The wire for any circuit needs to be thick enough to carry the necessary CURRENT (amps) without heating up. The voltage is represented by pressure and amperage is the volume of electrons, likely just the movement of component ct4t12 states, through the wires, while resistance is the tendency of the component ct4t12 states to stay within the conductor atom's matrix. I=V*R in this case means that the movement of ct4t12 states=V the number of ct4t12 states/tendency of those ct4t12 states to be held to the atomic matrix. Under AuT, R may be lowered or raised by bringing in or taking out ct4t11 states which exist around ct4t12 states because of staged compression, their presence would lead to the dissolution of the ct4t12 as is the case for all higher compression states.

17. Battery Design

According to the rule Planck E(photon)=hf (Planck constant×frequency) suggests photonic energy in a photon can be matched with any other frequency even if the particle size is not the same which allows the use of photons for this purpose.

FIG. 11 shows an exemplary design.

KFE features can be used to improve battery design. The idea here is that the primary elements of Li battery charging are dissolution of electrons to create a Li-ion which is more easily moved, and efficient stacking of the LI-Ion at the “negatively charged” ANODE during charging by non-random deposition which can both be accomplished with streams of ct4t11 inserted at strengths and locations determined to be fractally relevant by AuT Antennae strategically placed for this purpose within the battery matrix.

Charging is done with direct current, one concept is to provide current through wires 585 on either side of the AuT antennae biased with KFE to supply primarily ct4t11 drawn into the solution (not shown) of the battery through carbon antennae 578 opposite ion wires 587, doped with potassium, for example, within the semiconductor matrix 586 of the battery to pull the ct4t11 states through the carbon antennae.

An AC circuit could be used to pull in ct4t11-12 states sequentially into different areas of the battery matrix to maintain the dissolution and encourage the desired circulation of the Lithium ions.

Coiling the carbon antennae 578 and possibly internal wires 579 can be used to increase the amount of ct4t11 delivered relative to electric charge and may be oppositely charged to the proximate anode or cathode to increase the flow between the two which is less than the charging current for the battery. AuT insulators 575 can also be used to control the delivery effect between carbon antennae and internal wires 579.

Spiral alignment along spiral lines 214 for different components and here there is an anode ring 576 of carbon antennae, an outer ring 581 of carbon antennae, a separator component antennae 580a in the separator 580 and cathode antennae 583a close to the cathode 583 to control the flow of ct4t11 states.

Following AuT design with KFE using spiral layout, relative size (2\hat{ }n, spiral or both); separation of submatrix within the battery matrix (shown here with submatrices of (1) the cathode, (2) the anode, (3) an intervening layer between the two, and the other methods suggested by KFE can all be used. Spiral charge inward or outward towards the anode or cathode along preferably spiral pathways like spiral 214 towards the anode. Stepped charges at different locations is envisioned toward or away from area where buildup of ions is desired using ct4t11 states based on the amount of pretime change, size of delivery (branch or main line with or without breaks to spread out waves) which work to dissolve and solidify depending on the target, the outer electrons of Lithium shown by the number, size and spiral layouts of the carbon antennae including a dispersal array used to dissolved those atoms which would otherwise be forming dendrites.

Spiral charging and discharging (movement of the location where ct4t11 are put in or changing the movement of ct4t11 once it is inserted) can also be used to keep electrolytes circulating, disperse and limit dendrite formation or encourage dendrite dissolution. Balancing can be used on either side and on top and bottom of cathode/anode.

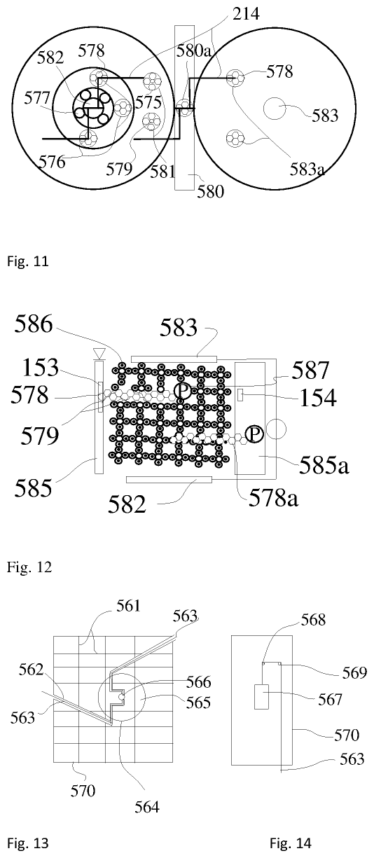

FIG. 12 shows a side view of the battery of FIG. 11.

Drawing the type and controlling the time of information into the battery substrate for holding charge, using carbon antennae charged by a charging means can improve the battery function although it might require programming to activate and deactivate the various items carrying the information.

In this view magnets 153 and 154 which are preferably small electro-magnets can be seen to deliver pulsed, alternating repulsive or attractive magnetic force in conjunction with or without all of the features shown in FIG. 10. While it is expected that repulsive magnetism would be best and less than and contrary to the charge of the anode or cathode in proximity because of the solvent properties of ct4t11 states sought, this would be subject to experimentation.

21. Composite Shear

The process of claim 1 further comprising including fractally significant spiraling during the cure process in at least three dimensions within a polymer layup during the cure process to extend three dimensional effects within the resulting mold.

Atomic fractal design shows that lower ct states, including simple atoms, tend to bond in two dimensions and to eliminate the resulting shear in the third dimension, higher dimensional effects need to be integrated into these same molecular bonds in two dimensions to give them more dimensional effects.

Resins tend to bond in two dimensions which ensures that shear forces will be uneven. Through rotation gravitational effects can be minimized; but the nature of the polymers is two dimensional. While overlap and balance (about a mathematical fulcrum), folding and unfolding are ways to include three dimensional effects, if allowed to cure without some outside force, the shears will be present. Over a significant area, spiraling can be introduced to form a three-dimensional cure within the mold, what can be referred to as a fractally significant spiraling within the mold during the cure process.

This can be done by stirring the resin, weave, or both during the curing process with the greater the stir in multiple directions, the less the resulting shear although this must be balanced with allowing the resin to set as fully as possible. Minimizing the amount of resin disturbed is one way to minimize the defects possible in this process.

In FIG. 13 adding dimension to polymers can be done using mechanics and chemistry within the layup weaves 561 of the same or alternate (e.g. cellulose) in 3 dimension; done during curing by stirring mechanically during the early curing process and then stopping, in this case by using lines 563 pushed or drawn preferably through the resin (not shown) creating spirally relevant forms 562 in 3 dimensions through the mold. This is accomplished by winding then unwinding during the cure the lines 563 around hardened areas 565 in the weave to guide the lines 563 along desired pathways to leave the forms 562 in the cured resin.

It could be done by extruding those dimensional features necessary to a fractally significant spiraling (balance, overlap and folding) within the curing structure. The width of a string is not the issue, so it could be one by pulling sheets 567 which are preferably made up of absorbent threads using lines 563 around pivots 568 and 569 to change the dimensional direction of travel of the sheets 567 to get the effects desired. This could be done with magnetic particles within the matrix using magnetism in place of the lines 563 and pivots 568 and 569.

FIGS. 13-16 shown ways to encourage three-dimensional fractal compression forms to form within a weave of the composite fractal extensions in all dimensional where you want to reduce shear utilizing the features of the elements of fractal construction in multiple dimensions (overlap, folding, and balance) along fractally relevant lines “in the resin” maximizes the effect.

This is preferably done throughout the mold, but particularly in the areas where shear is experience and at different points and different angles to prevent weakness in one area and in one dimension.

The same process can be used to incorporate electronic features tied to fractal designs into the matrix of a curing polymer.

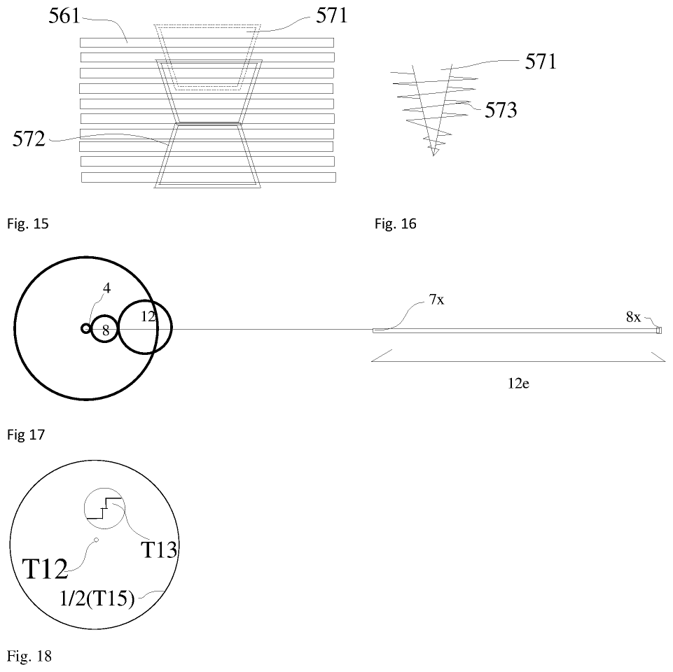

FIGS. 15 and 16 show the use cellulose porous plugs 571 being pushed during curing through weave 561 into a mold 572. These may be twisted during the curing process to get vertical strength with polymers is another technique.

The goal is to get two-dimensional polymer structures to buckle into a third dimension, folding of multiple layers to mimic fractal folding.

Vinyl and cellulose composite atomic modeling of AuT chemistry allow the targeting of shear and electromagnetic properties. The combination of vinyl resin and cellulose based products allow the structure features of both to be used with partial or complete treatment and curing.

Drawing these into the mesh against each other while twisting them with the varying widths or threading or one through the other in this fashion during the curing process, can create the type of balance, overlap and folding needed to get the effects.

Dual or Single resin activation is considered, where different resins or reactants are used for bonding of the main weave and the insert. In FIG. 16 a screw 573 is shown which can be in place of the plug and mold which screw 573 would also be porous.

Hemp/rope materials of cellulose can be used as a method of enforcing the cellulose weave being drawn through spirals in and out of the primary weave of the layup.

Titanic Events

AuT defines higher compression states, particularly black holes which explains why critical large-scale events occur, namely earthquakes, super volcanos and the shift of the earth's magnetic fields, issues critical to the survival of mankind. If we are moving through a large wave, then eventually the wave form will reach zero and go opposite shifting the earth's core with catastrophic effects.

FIGS. 17 and 18 Show Equivalence Vs. Actual Size

Fractal equivalence from a post-time, energy perspective is differentiated from actual size. If an atom was collapsed, a zero-energy state in terms of thermodynamics or a perfect fractal in the language of AuT, Atoms might look like FIGS. 8-e, but Atoms are dynamic because they are absorbing and spewing information. Atoms have expanded backbones, cores and electron shells separated from the nucleus by large distances.

Information quantity compared to locational quantities is shown in FIG. 17, showing the relative size of particles and occupied orbits with the neutron 4, proton 8 and electron 12 being shown, but with the effective area occupied by the electron 12e being between 2\hat{ }7 and 2\hat{ }8 the neutron area.

The electron pair t13 is 5.4 10\hat{ }12 states and assuming the same doubling effect, ×2 for 15, ×4 for 14, the electron would occupy space 16 times that of the neutron; but from a time based perspective, this larger space would might be reflected in ×16 locations.

There are 3 separate measurements of any particle from a thermodynamic standpoint; 1) information content, 2) size, 3) locational area, this last also being affected by the amount of pretime change.

Informational changes between states involve a post-time analysis as shown in FIG. 18. Here a t12 state is compared with an electron pair t13 for size relative to ½ of a proton t15 in terms of information. The individual t12 state can occupy more area than the ½ T15 state during any measurable time period because the t12 state is largely pretime independent in its movements although a finite number of quantum changes in the quantum count occur during any measurable time period.

Exemplary Molecules

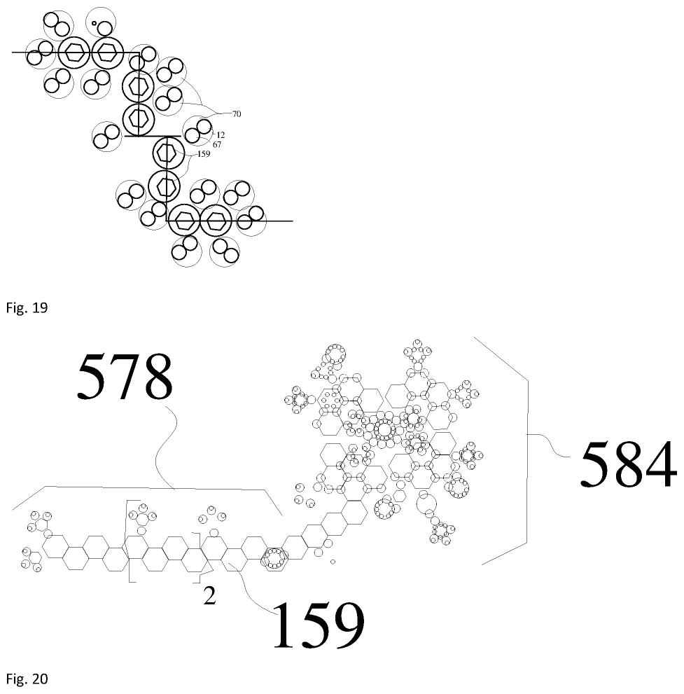

FIG. 19 shows an octane atom, the hydrogens 70 are on the outside of the carbon hexagons 159, react quicker as a result, turning into water, absorbing heat and expanding. The alignment shown along spiral arms is reflected for all molecules including complex molecules like DNA which would mean that the helix is developed around overlapping spirals. Another process is separating particles within a matrix that have more or less pretime change (e.g. within the fuel) which allows dead end reactions to be minimized Fractal design energy release from atoms

AuT atom design shows that energy is released from reactions from the rearrangement of chemicals to require less low information (pre-electron) states which are released as energy. Released pretime states have different units of change. Expansion and collapse according to fractal compressive features can be balanced according to internal higher compression states balanced by successive less compressed states targeted to maximize the release of pretime change at different levels (ct4t11-12 being the most likely) and to minimize the release of undesirable results.

Atoms form according to fractal patterns, the formation of carbon monoxide and carbon dioxide in burning carbon fuels historically ignore the fractal structures possible in carbon chemistry which can be targeted based on trapping carbon using KFE including low electron shell “area/volume” ct4t11 footprints in the resulting molecular structures thereby releasing more energy.

No one has ever designed reactions or reaction chambers to target the KFE features of dimension because these features of the atom were not previously identified.

Another possible target is to remove and capture Hydrogen released from the reactions or to have it react in a more efficient manner than merely producing water or at least to maximize the energy released by the H2O reaction using the same structural changes targeted for the carbon reactions.

Increasing the energy held within the Oxygen to H bonds is a process which can be maximized using KFE. The loose H from another water expands them and gas can be maximized in terms of pressure by targeting the transition between the energy held in the other bonds and the energy already existing and created during burning of the bonds to create water and to otherwise release energy. Targeting the KFE involved at each stage of a combustion process using water expansion allows the water to be formed, energy generated and the absorption of the energy as expansive heat in as efficient a manner as possible.

If you take the structure of the atom as shown in the NPTE you can see why gasoline works so well as a pressure-based fuel. Octane burning generates an exterior shell of water atoms produced in a high concentration gas around the formation of Carbon Oxygen compounds, the water and expanded to provide pressure to push a piston (typically). Although it exists only for a short period of time, both the water shell and the internal carbon reactions can be manipulated using the new fractal atomic design and other KFE features to maximize the burn of the fuel and the expansion of the water atoms, to capture and convert long wave pretime change

The individual steps in the octane-oxygen reaction include sequentially, the generation of water atoms, the generation of carbon-oxygen atoms, the resulting release of pretime ct4t11 states with (for purposes of the reaction) primarily heat wavelength absorption as the absorption by AuT antennae in the water of ct4t11 states resulting in their rapid expansion and lower compression fractal states as gas phases.

Adding ethanol or some other water significant atomic mixture can change the fractal structures to maximize how much of the heat goes towards expansion and how much is lost not only with octanes and hydrogens, but with solid fuels such as boron. The type of fuel, the relative contents of the fuel during the burning process, the timing of the fuels, volumes and the control of the energy states all of which can be controlled using AuT processes and mathematics to increase the efficiency of energy release.

Traditional engines look only at abstract energy, AuT allows for time to be taken out of the equation and for focus on how to use KFE to maximize the wavelengths (amount of pretime change) of the ct4t11 states, and AuT antennae in the surrounding higher ct states to maximize the type and location of pretime states separating and combining with atoms and molecules through KFE to control pressure, energy types and reactants generated. FIG. 20 shows a carbon antenna 578 occurring in nature as the long arm of a chlorophyll molecule with a charging molecule segment 584.

AI

All AI is a function of algorithms and at their base is the same math that gives rise to all other things, adapted as shown in the various applications here, for the particular use at the scales and rates of change that are applicable.

Facial, body recognition; but more importantly prediction tied to alignment and choices inherent in the dual, overlapping spirals at different levels of compression targeting fractal features according to the math of AuT. Computational programming is tied to algorithms. While applications are more varied and diverse rendering AuT irrelevant to most, when you focus on “basic research aimed at having the broadest possible impact, the development of computational methods should include an emphasis on theoretical underpinnings, on rigorous convergence analysis, and on establishing provable bounds for approximation methods” you are ultimately getting to a foundation which is defined by AuT in terms of practical applications as opposed to the equally important human applications. Practical applications, the movement and interaction of both large- and small-scale phenomena are AuT centric determinations. Very quickly programming goals get away from this area, but at their foundation, this science remains pervasive.

It is not narrowly applicable. All systems in the universe break down into fractal components defined by the math of AuT. Energy is a function of the pre-time change at the ct4t12 level which results from and is made continuous by the underlying changes within that state. Materials and their interaction occur along the lines of folding defined by the overlapping spiral design of AuT manifesting itself at each level of exponential compression. Mathematicians have long looked for symmetries in changing material states. AuT provides the math for this, showing that as molecular fractals breakdown, they do so into or in association with lower order AuT fractals. One example is Lithium breaking down an electron bond, essentially liquifying the bond with an abundance of ct4t12 states so that the structure more closely resembles that of hydrogen so that it may move through a semiconductor allowing a lithium-ion resulting to be charged or discharged.

While AuT changes are called “compression” and “decompression,” it is also, to use the language of this section, turbulent and bi-directional, at scales determinable more accurately and far below that possible with other models.

Processes include using the non-pretime change for consistency and pre-time to move or as morse code to convey information, pre-time changes to get more instantaneous communications, ct2-ct3 for example, to move communications faster than time, pretime changing with non-pretime changing or amount of pretime change.

Notes on Key Fractal Elements

KFE includes fractal difference in dimension which are themselves a fractal result of focus so that the denser, the more dimension.

KFE is focusing on the spiral features, the compression features, the changing vs stable pretime features, the size of the time and pretime features (the ct states involved); the nature of pretime change vs time-based change, the relative amount of pretime change vs non change in the matrix being mined for changing information, the method of mining changing information or a combination of those features.

KFE include: Staged (1, 2, 3, 5 or 2, 4, 8, 16 or both overlaid-see chemistry form) (increase or decrease) size of spark, heat, temperature, timing or time from plug or pressure from the chamber during the burn; Staging can be, for example, 3 in a row, stepped, different positions in burn, forward, backward or alternating forward and back.

KFE includes holding of the time, pressure, heat, or spark is a part of the timing. A four-stroke engine may have the fractal features of the different strokes may be treated aas KFE.

KFE includes staged conversion in the same terms is a part of staging; Staged fuels (1:1; 1:2: 1:3:1:5 one type of additive to the next); burn times, heat, pressures, mixtures, strokes (e.g. 2, 3, 5) can be staged to get fractal results. Controlled Water content for maximum expansion is one target for efficiency given the role AuT shows of water expansion in fuel burning.

KFE includes the fractal design of atoms including the Neutron backbone and the structure of resulting proton cores and electron clouds as pathways to increase and control the release of energy, a greater absorption of the energy in terms of expanding the water matrix (increasing the pressure); and to have resulting carbon atoms that are more easily trapped after the reaction.

KFE includes Movement of reactants can be maximized directionally using alignment or net alignment of the fractal elements, particularly with solid fuels, but the same processes can be used for other fuels reflected using six sided cylinders in cars or other fractal modeling as chemistry or mechanical design or both.

KFE includes the rate of injection, sequential fuel transitions, handled by bundling the fuel chemically. This may be done with shaping of the reaction chamber parts (cylinder, piston, injectors) with the number, location, and series of injector jets and by controlling the mixtures of the elements.

KFE includes Shape of pistons, fractal structures at gross and atomic levels can be used to make sure that heat energy goes to expansion.

KFE includes the type of molecules created, and the orientation of atoms created the order of creation and the orientation and energizing of atoms, and even the parts of those that are energized according to the use of KFE elements. This distinguishes AuT in terms of precision, the ability to target the elements giving rise to energy and their interaction with higher compression features, tying photons to atomic structure to use antiquated terms of pre-AuT physics; more precisely, pretime change and t11 states to ct4t12 transition electrons and the matrix between those and the proton core balancing the neutron backbone of the atoms in question.

KFE targets include alignment, quantities, for example the 3:1 alignment of location (shown in Fig. Neon, for example), using KFE to target pressure and/or volume, for example: Heat to flame 1, 2, 3, 5; or 2 4 8 16 exponentials to target the fractal elements of the detonations or the pauses in between.

Key elements of Carbon Capture and Conversion are: 1) focusing on the resulting release of low energy states from fractally defined collapsed ct states and 2) the fractal design of carbon (ring) structures inherent in KFE chemistry modeling; and 3) using KFE to selectively generate and capture desirable ct states.

1. A process for dimensional manipulation comprising the steps of (1) changing one or more key fractal elements as features of at least one ct state within at least one AuT matrix comprised of a plurality of ct states where ct states are sequentially, fractally compressed information making up dimensional features.

2. The process of claim 1 wherein the at least one AuT matrix is comprised of dimensionally sequential ct states within the at least one AuT matrix of at least one highest ct state compression level; but allowing that external lower ct states with lower compression than the at least one highest ct state compression level, might be dimensionally sequential with ct states of the same compression level within the at least one matrix, but also external to the at least one matrix.

3. The process of claim 1 wherein dimensional manipulation is further defined as being from one or more of the following: design of the at least one AuT matrix, performance prediction of at least one AuT matrix, change net features or arrangement of ct states in at least one AuT matrix, combine the at least one AuT matrix with at least one second AuT matrix, categorize the at least one AuT matrix, extract energy relevant ct state features from the at least one matrix to at least one second matrix, store energy relevant ct state features within the at least one matrix, transfer energy relevant ct state features from at least one first AuT matrix to at least one second AuT Matrix; computing using time and pretime features within the at least one AuT matrix; determining structural changes by looking from the perspective of different ct states; manipulating spiral or compressive features of the at least one matrix; creating balance or imbalance within the at least one first matrix with ct states from at least one second AuT matrix by a) using ct states with different degrees of pretime change, b) exchanging ct states between the at least one first matrix and the at least one second matrix to create contact, c) changing compression and decompression about AuT fulcrums, d) maximizing the efficiency of exchanges of ct states between the at least one first matrix and at least one second matrix; e) creating fractal alignment between ct states associated with the resulting ct state matrix sought; f) changing net absorption and spew to get separation, attractive or repulsive effects; identifying the et states which are to be manipulated; selecting a compression or decompression ct state component to change the selected ct states; adding compression or decompression components to yield the new ct states; 10) modifying at least one AuT fulcrum.

4. The invention of claim 1 wherein key fractal elements (KFE) are (1) stepped AuT fractal transitions defined as fractal transitions governing ct state changes; 2) fractal balance, defined as alignment of at least two higher compression ct states about at least one AuT fulcrum comprised of lower compression states about which higher compression ct states share information; and AuT fulcrums defined as lower compression ct states at the overlap of compression of at least two higher compression ct states; balancing with lower ct states a ct4 state neutron backbone; 4) absorption of ct states (absorption) towards compression and spew of ct states (spew) towards decompression within at least one AuT matrix or between multiple AuT matrices; 5) f-series spirals of ct states in and out of alignment for absorption and spew; 6) pairing of higher compression ct states along f-series linear spirals about shared lower compression ct states; 7) folding to get compression and unfolding to get decompression along fractal linear spirals of ct states about at least one AuT fulcrum, 7) compression as lowering the amount of lower compression ct states between higher compression ct states and decompression as increasing the amount of lower compression ct states between higher compression ct states; 8) fulcrums as AuT plasma centers between ct states defined as the areas between ct states where lower ct states are shared as at least one of compressive or decompressive results; 8a) net compression or decompression as force when observed from the standpoint of time; 9) ct states defined as stepped (golden ratio) fractal dimensional states from common iterated equations defined by fractal compression or decompression due to compression of lower ct states along f-series fractal lines; 10) force defined as the result of net winding or unwinding of ct states as viewed from post time ct state perspectives including ct4t11 changes viewed as energy; 11) time defined as stop frame animation resulting from changes in pre-time ct states defined as ct states below the level generating electromagnetic effects; relativistic effects as the difference between pretime and time based change; 12) using AuT as “base logic” of at least one AuT Matrix; 13) net compression as the net compression or decompression within an AuT matrix; 14) shifting between higher and lower compression of ct states within the AuT fulcrum; 15) absorption and spew between at least the first AuT matrix and at least one second AuT matrix; 16) fuse length as a fractal element of ct state transition from compression to decompression; 17) net AuT compression as manifested at different ct states, 18) ct state exchange between at least two AuT Matrices in place of collision or field modeling; 19) AuT matrix categorization using 1) ct state content, 2) amount of ct states within the content; 3) relative dimensional size to at least one second matrix, 3) locational area from the perspective of time of generated by pretime change of the at least one matrix; 4) AuT plasmas; 5) fulcrum locations; 20) basing thermodynamic effects of at least one aut matrix based on categorized ct states within the at least one aut matrix; 21) treating EXCHANGE of lower ct states between at least two higher ct state AuT matrices as the source of interaction; 22) proton positron atomic links for holding electrons; 23) collisions as the exchange of information between at least two AuT matrices; 24) post collision effects reflecting the net change of ct state and pretime change in each matrix of the at least to matrices; 25) targeting fulcrums and stepped transitions; 26) quantum fractal dimensional change resulting is quantum time; 27) fulcrums as shared ct states between higher ct states; 28) curvature defined by a solution to fpix for pi with definitive limitations generating the sequential amounts of dimension and curvature in response to net ct state compression.

4. The process of claim 1 wherein key fractal elements (KFE) are stepped transitions for transfer and interaction of information according to the definition that one ct state only directly interacts with a like group of ct states reflecting one change in the quantum count, but because of different fuse lengths, many of these changes for states folded together (above ct1) change simultaneously due to common fuse termination, fuse termination defined as the point when the state of a ct state changes due to the net solutions being positive or negative as defined by the sum of the individual fuse lengths of individual ct1 states.

5. The process of claim 1 wherein the at least one matrix has molecular structure and wherein the process further comprises changing molecular structure as a fractal using abs and spew from at least one second matrix targeting absorption and spew from ct states within the at least one matrix.

6. The invention of claim 1 wherein compression between ct states is balanced and wherein balance is defined by the elements of a fulcrum of lower states about which higher states balance through absorption and spew and at least one KFE approximately aligned on either side of the fulcrum.

7. The process of claim 1 further comprising reconciling traditional EMT with AuT quantum dimensional change to do at least one of the following: improve time keeping, energy regulation and energy control.

8. The process of claim 1 further comprising taking quantum change in dimension to scale an absolute value or a best usable value of “Planck or Electromagnetic” time (PT or EMT).

9. The method of claim 1 wherein the juxtaposition of at least one of odd and even exponent states and pretime and post time states are used for dimensional manipulation.

10. The method of claim 1 wherein manipulation comprises treating magnetism and electricity as features of compression and decompression.

11. The invention of claim 1 comprising manipulating ct state compression through balancing and unbalancing an wherein balance is defined by the KFE of higher compression ct states about a fulcrum of lower compression ct states.

12. The method of claim 11 further comprising maximizing desired neutron balancing and unbalancing to stabilize or destabilize desired proton area sharing with fractal folding at different ct state compression levels.

13. The process of claim 1 comprising absorbing and deflecting Radiation utilizing interaction of lower level ct states within a ct4t11 state of radiation with lower ct states within an AuT antennae in an AuT matrix.

14. The process of claim 1 comprising balancing a neutron backbone about at least one AuT fulcrum stabilized by a core of protons.

15. The process of claim 1 comprising balancing a ring of ct4t15 states about at least one AuT fulcrum to collapse a proton into a neutron.

16. The process of claim 1 further comprising maximizing energy from waves utilizing KFE, features of the wave and absorbing ct states for at least one of the following: matching information sharing; targeting ct states to be drawn out from the absorbing atoms; and targeting ct states to be drawn from radiation.

17. The process of claim 1 further comprising including fractally significant spiraling during polymer cure processes in at least three dimensions within a polymer layup.

18. The process of claim 1 further comprising manipulating at multiple compression states.
